(19) United States
(12) Patent Application Publication
(10) Pub. No.: US 2022/0323444 A1
(43) Pub. Date: Oct. 13, 2022

(54) Methods of Treating Advanced Prostate Cancer

(71) Applicant: The Medical College of Wisconsin, Inc., Miwaukee, WI (US)

(72) Inventor: Marja Nevalainen, Milwaukee, WI (US)

(21) Appl. No.: 17/631,366

(22) PCT Filed: Jul. 29, 2020
(86) PCT No.: PCT/US20/44002

§ 371 Date: Jan. 28, 2022

Related U.S. Application Data
(60) Provisional application No. 62/879,708, filed on Jul. 29, 2019.

(51) Int. Cl. A61K 31/519; A61K 31/4188; A61K 31/4439; A61K 31/506; A61P 13/08; A61P 35/04

(52) U.S. Cl. CPC A61K 31/519; A61K 31/4188; A61K 31/4439; A61K 31/506; A61P 13/08; A61P 35/04

(57) ABSTRACT

The present invention relates to methods for treatment of prostate cancer, including castrate resistant prostate cancer that resistant to at least one anti-androgen therapy. The methods include administering an effective amount of a Jak2 inhibitor which inhibits through the Stat5 pathway and is able to reduce or inhibit expression of androgen receptor in prostate cancer.

### CROSS-REFERENCE TO RELATED APPLICATIONS

This patent application claims the benefit of priority of U.S. Provisional Patent Application No. 62/879,708, filed Jul. 29, 2019, which is incorporated herein by reference in its entirety.

### STATEMENT REGARDING FEDERALLY SPONSORED RESEARCH

N/A

### BACKGROUND

Men diagnosed with prostate cancer (PC) at an advanced stage or displaying tumor recurrence after surgery require androgen deprivation therapy (ADT) to inhibit the androgen receptor (AR) transcription factor(1, 2). Androgen deprivation therapy (ADT) is the predominant treatment for advanced prostate cancer (PC). A major challenge in the clinical management of advanced PC is the progression of the disease to lethal castrate-resistant PC (CRPC) in virtually all patients. In the majority of cases, CRPC results from a failure of ADT to maintain durable suppression of androgen receptor (AR), which is the molecular target of ADT. AR is the most frequently altered gene in the CRPC genome, AR is persistently expressed in the nucleus of the majority of the cells in CRPC tumors, and serum levels of the AR target gene, PSA, continues to rise in these patients.

More potent second-generation AR antagonists (enzalutamide, ENZ; apalutamide, APA; darolutamide, DARO; abiraterone, ABI) were developed to re-target the persistent AR activity in CRPC tumors and have become standard-of-care in this setting. These drugs have been shown to more effectively antagonize the AR ligand-binding domain (ENZ, APA, DARO)(3) and reduces androgen synthesis (ABI)(4). However, despite the initial promise, CRPC remains a uniformly lethal disease due to a rapid development of resistance to these second-generation drugs. For instance, due to this resistance, ENZ provides an improvement in survival of PC patients only by 4-6 months. It has been determined that AR is still expressed and remains active in the majority of CRPC and drives resistance and continued tumor growth, ultimately causing patient death(5).

Therefore, there is a need for therapeutics that target the persistent AR expression and AR transcriptional activity in advanced PC, and in CRPC after the conventional anti-androgens fail.

### SUMMARY OF THE INVENTION

In one aspect, the present invention provides a method of inhibiting and reducing expression of androgen receptor (AR) and androgen receptor variants (AR variants) in a prostate cancer cell, the method comprising: contacting the prostate cancer cell with an effective amount of a Jak2 inhibitor which inhibits through Stat5 pathway within the cell to inhibit or reduce AR and AR variant expression in the prostate cancer cell. In some aspects, the Jak2 inhibitor is selected from the group consisting of pacritinib, gandotinib, baricitinib, and fedratinib. In some aspects, the prostate cancer is selected from the group consisting of androgen-sensitive prostate cancer, advanced prostate cancer and castrate resistant prostate cancer (CRPC).

In another aspect, the present disclosure provides a method of inhibiting or reducing growth of prostate cancer (PC) in a subject having PC, the method comprising administering a therapeutically effective amount of a Jak2 inhibitor that acts through the Stat5 pathway selected from the group consisting pacritinib, gandotinib, baricitinib, and fedratinib to inhibit or reduce growth of the PC cells within the subject.

In another aspect, the disclosure provides a method of inhibiting anti-androgen resistant CRPC cell growth in a subject with prostate cancer, the method comprising administering an effective amount of a Jak2 inhibitor that inhibits through the Stat5 pathway to the subject with prostate cancer in order to treat the cancer and inhibit growth of anti-androgen-resistant prostate cancer cells.

### BRIEF DESCRIPTION OF DRAWINGS

FIGS. 1A-1C is a cartoon of androgen receptor (AR) gene organization. Cryptic exon 3 (CE3) is red. Exon organization (FIG. 1A) and protein domain structures of (FIG. 1B) full-length AR and (FIG. 1C) AR-V7 are shown. N-terminal domain (NTD); DNA binding domain (DBD); hinge (h); ligand binding domain (LBD).

FIGS. 2A-2D demonstrate the role of Stat5 in AR expression in androgen-sensitive and castrate-resistant prostate cancer (CRPC) cells. FIG. 2A. Stat5 knockdown robustly inhibits AR mRNA expression in both androgen-sensitive and CRPC cells. Stat5a/b was inhibited by lentiviral expression of Stat5a/b shRNA (shStat5a/b) or non-target shRNA (shCtrl) in 6 different PC cell lines for 72 h, followed by RNA extraction and quantification of FL-AR, AR-V7 and AR-V9 mRNA levels by qRT-PCR. FIG. 2B. Time-course of AR mRNA suppression by knock-down of Stat5 in PC cells. Stat5a/b was inhibited by lentiviral Stat5a/b shRNA (shStat5a/b) or non-target shRNA (shCtrl) in PC cell lines for the indicated time points, followed by qRT-PCR of FL-AR, AR-V7 and AR-V9 mRNA FIG. 2C. Stat5 drives AR protein expression in PC cells. Immunoblotting (IB) of AR in PC cells 72 h after lentiviral expression of Stat5 shRNA (shStat5a/b) vs. control shRNA (shCtrl). Effective genetic depletion of Stat5a/b and equal loading are demonstrated by IB with anti-Stat5a/b and anti-actin antibodies of whole cell lysates (WCLs), as indicated. FIG. 2D Stat5 induces directly the transcription of the AR gene in prostate cancer cells. Inhibition of new protein synthesis by cycloheximide (CHX) does not block induction of AR mRNA levels by active Stat5 suggesting that Stat5 regulation of the AR gene expression is not mediated via another protein. Constitutively active Stat5 (CAStat5) was expressed by lentivirus in CWR22Rv1 cells followed by treatment of the cells with 10 μM CHX for 24 h.

FIG. 3 demonstrates that knock-down of Stat3 does not affect AR mRNA expression in PC cells. Stat3 was inhibited by lentiviral expression of Stat3 shRNA (shStat3) or non-target shRNA (shCtrl) in PC cell lines for 72 h, followed by RNA extraction and quantification of FL-AR, AR-V7 and AR-V9 mRNA levels by qRT-PCR with lenti-shStat5 infected cells as controls.

FIGS. 4A-4B demonstrate the effect of Stat5 inhibition of AR levels in human PC xenograft tumors grown in nude mice and in patient-derived clinical PCs ex vivo. FIG. 4A. Pharmacological Stat5 inhibition decreases AR protein and mRNA levels in PC xenograft tumors in nude mice. Nuclear AR and Stat5 levels were detected by immunostaining of paraffin-embedded tissue sections of CWR22Pc tumors from mice treated with IST5-002 (IST5) (40 mg/kg; i.p.) for 40 days. Also, AR mRNA levels were evaluated by q-RT-PCR. FIG. 4B. Pharmacological inhibition of Stat5 by IST5-002 or pacritinib suppress AR mRNA levels in patient-derived PCs cultured ex vivo. Clinical PCs (n=4) obtained from prostatectomies were cultured for 7 days ex vivo in 3D tumor explant cultures in the presence of Stat5 inhibitor IST5-002 (12.5 μM) or PAC (1 μM) vs. vehicle, and AR mRNA levels were analyzed by qRT-PCR. This data show that Stat5-regulation of the AR levels is not limited to prostate cancer cell lines cultured in vitro, but also holds for human PC xenografted to nude mice as well as to patient-derived clinical PCs cultured ex vivo.

FIGS. 5A-5B demonstrates that active Stat5 induces/upregulates AR mRNA and protein levels in both androgen-sensitive and CRPC cells. FIG. 5A. Active Stat5 induces a robust increase in AR mRNA levels in PC cells. Stat5a/b signaling was increased by lentiviral expression of constitutively active (CA) Stat5a/b or GFP (control) for 72 h in both androgen-sensitive and CRPC cell lines followed by RNA extraction and quantification of FL-AR, AR-V7 and AR-V9 mRNA levels by qRT-PCR. FIG. 5B. Active Stat5 induces a robust increase in AR protein levels in androgen-sensitive and CRPC cells. Stat5a/b signaling was increased by lentiviral expression of constitutively active (CA) Stat5a/b or GFP (control) for 72 h in PC cell lines followed by IB analysis of AR protein levels. Increased active Stat5a/b levels and equal loading are demonstrated by immunoblotting with anti-Stat5pY mAb and anti-actin pAb of WCLs.

FIG. 6A demonstrates the ability of Jak2 inhibitors that act through the Stat5 pathways to suppress AR mRNA levels in PC cells. Momelotinib, a Jak2 inhibitor that does not work through Stat5, does not suppress AR expression in PC cells. Thus, it is specifically the pharmacological Jak2 inhibitors that act through Stat5 to suppress AR-FL and AR-V mRNA levels in PC cells. Jak2 was inhibited for 3 days by Jak2 inhibitors AZD1480, gandotinib, pacritinib, baricitinib or fedratinib at indicated concentrations followed by RNA extraction and quantification of FL-AR, AR-V7 and AR-V9 mRNA levels by qRT-PCR.

FIG. 6B demonstrates pharmacological inhibition of Jak2 through Stat5 signaling potently suppresses AR protein levels in both androgen-sensitive and CRPC cells. Again, Jak2 inhibitor momelotinib, which does not act through Stat5, did not result in the suppression of AR protein levels within PC cells. Immunoblot analysis of AR expression in PC cells treated with increasing concentrations of Jak2 inhibitors AZD1480, gandotinib, pacritinib or baricitinib for 72 h. Alternatively, cells were treated with the Stat5 inhibitor IST5-002 (IST5) at indicated concentrations, followed by IB analysis of AR, active Stat5a/b (pYStat5) and Stat5a/b with actin as a loading control.

FIGS. 7A-7E demonstrate that gandotinib (FIG. 7A), pacritinib (FIG. 7B), and fedratinib (FIG. 7C) are potent inducers of death of CWR22Rv1 PC cells with IC50 of 300 nM at 3 days, while momelotinib does not provide the same effect of beneficial induction of cell death (FIG. 7D). Moreover, another Jak2 inhibitor, Ruxolitinib (RUXO), does not induce death of PC cells (FIG. 7E).

FIG. 8 shows that active Stat5 levels were robustly elevated in PCs from patients treated by ENZ compared to hormone naïve PCs, demonstrated by immunostaining of paraffin-embedded tissue sections with anti-Stat5PY mAb (magnification 40×).

FIGS. 9A-9C demonstrate that ENZ induces Stat5a/b activation in PC. FIG. 9A. CWR22Pc and LAPC4 PC cells were treated with ENZ or vehicle for the indicated periods of time. Levels of active Stat5 were determined by immunoprecipitation (IP) of Stat5 followed by immunoblotting (WB) for pStat5a/b and total Stat5. Whole cell lysates (WCL) were immunoblotted for pStat3, Stat3 and Actin. FIG. 9B. Stat5 phosphorylation levels are elevated in ENZ-resistant (R) (CWR22Pc AR-F876L) cells compared to parental CWR22Pc cells (right panel). FIG. 9C. AR is required for ENZ induction of Stat5 phosphorylation. CWR22Pc and LAPC4 cells were transduced with lentiviral AR shRNA (shAR) or lenti-shCtrl for 3 days followed by treatment with ENZ or vehicle for 7 days at the indicated concentrations.

FIGS. 10A-10D demonstrate that ENZ induced Stat5 activation in PC occurs through Jak2. FIG. 10A. PC cells were cultured with ENZ or Jak2 inhibitor AZD1480 (800 nM) alone or in combination for 12 days. Expression levels of active Stat5 were determined by IP of Stat5 followed by WB for pStat5a/b and total Stat5. WCLs were immunoblotted for pStat3, Stat3 and Actin. FIG. 10B. Genetic knockdown of Jak2 blocks ENZ-induced Stat5 phosphorylation in PC cells. Jak2 was suppressed by lentiviral Jak2 shRNA vs. shCtrl in PC cells for 3 days followed by ENZ or vehicle for 7 days. Active Stat5, Stat5 and Jak2 levels were evaluated by IP and WB, as depicted. FIG. 10C. ENZ induced Jak2 phosphorylation in PC cells. PC cells were treated with ENZ or vehicle for 6 h at the indicated concentrations. Control cells were stimulated with prolactin (Prl) (10 nM) for 20 min as control for cytokine-induced Jak2 phosphorylation. FIG. 10D. Alternatively, PC cells were treated with ENZ or vehicle for 12 days or stimulated with Prl for 20 min at the indicated concentrations. Stat5 and Jak2 were IP:ed and WB:ed for pStat5, pJak2, tot Stat5, and total Jak2.

FIG. 11 demonstrates that knock-down of Stat5 suppresses ENZ-induced FL-AR and AR-V7 expression in PC. Stat5 was inhibited by lentiviral expression of shStat5 followed by ENZ treatment of the cells for 7 days. AR mRNA was analyzed by qPCR and protein levels by WB.

FIG. 12 is a schematic representation of the proposed ENZ induction of hyperactivated Jak2-Stat5 feed-forward loop in PC.

FIG. 13 demonstrates that ENZ induces the expression of the EMT markers in PC cells, which is blocked by genetic knockdown of Stat5. Treatment of CWR22Pc PC cells with ENZ (40 μM) for 10 d induced the expression of N-Cadherin, Twist 1 and Vimentin, and down-regulated E-cadherin levels in CWR22Rv1, LAPC4 and CWR22Pc PC cells. Lenti-viral expression of ShStat5 3 days prior to ENZ treatment (at indicated concentrations) blocked ENZ-induction of the EMT markers.

FIG. 14 demonstrates that ENZ induces migration of CWR22Rv1, CWR22Pc and LAPC4 cells, which is blocked by genetic suppression of Stat5. Stat5 was genetically knocked down by lentiviral expression of shStat5 with shCtrl as control for 3 days followed by treatment with with ENZ (40 μM; CWR22Pc, CWR22Rv1; 20 μM LAPC-4) vs. DMSO for 10 days. Equivalent numbers of cells were plated to Boyden chamber inserts with Fibronectin as the chemoattractant in the bottom well, and the migrated cells were imaged and counted.

FIG. 15 demonstrates that active Stat5 (CAStat5) induces a robust increase in PC metastases formation in liver after orthotopic growth of CWR22Pc as tumors. Constitutively active Stat5 (CAStat5) was expressed in CWR22Pc cells using lentivirus prior to inoculation of the cells to the prostate of NOD-SCID mice with GFP expressing cells as control. After 60 days, the mice were sacrificied and the livers were imaged.

FIG. 16 demonstrates that inhibition of Jak2 by fedratinib (FED) suppress AR-FL and AR-V and protein levels in CRPC cells. Jak2 was inhibited for 4 days by fedratinib (FED) at indicated concentrations followed by quantification of FL-AR, AR-V7 and AR-V9 protein levels by IB.

FIG. 17 demonstrates that clinical grade pacritinib (CTI) has higher efficacy than research grade pacritinib (MCE) in suppressing mRNA expression of both full-length (AR-FL) and variant (AR-V7 and AR-V9) forms of AR in the PC cell lines CWR22Rv1 (top panel) and CWR22PC (bottom panel). Control cells were treated with lenti-shJak2 (ShCtrl).

FIG. 18 demonstrates that clinical grade pacritinib (CTI) has higher efficacy than research grade pacritinib (MCE) in suppressing protein expression of both full-length (AR) and variant (AR-V) forms of AR in the PC cell lines CWR22PC (left) and CWR22Rv1 (right).

FIG. 19 demonstrates that mRNA expression levels of full-length (AR-FL) and variant (AR-V7 and AR-V9) forms of AR decrease with the increased concentrations of FED (top) and PAC (bottom).

FIG. 20 demonstrates that lentiviral overexpression of constitutively active (CA) Stat5 counteracts the inhibition of AR by PAC (left) and FED (right) at both the protein (top) and mRNA (bottom) levels in CWR22PC PC cells.

FIG. 21 demonstrates that FED (left) and PAC (right) do not further reduce AR protein (top) or mRNA (bottom) expression after genetic knockdown of Stat5 using lentiviral expression of Stat5-shRNA (shStat5) in CWR22PC cells.

FIG. 22 demonstrates that treating CWR22PC PC cells that survive treatment with ENZ with FED or PC reduces cell viability with high efficacy. The PC cells were treated with ENZ (or vehicle) for 5 days, and were then treated with ENZ, vehicle, PAC, or FED for 4 days.

FIG. 23 demonstrates that treating CWR22PC PC cells that survice treatment with ENZ with FED or PC reduces expression of AR at the mRNA (top) and protein (bottom) level. The PC cells were treated with ENZ (or vehicle) for 5 days, and were then treated with ENZ, vehicle, PAC, or FED for 4 days.

FIG. 24 demonstrates that PAC causes a reduction in AR protein levels in CWR22PC cells starting 48 hours after treatment, while genetic knockdown of Stat5/Jak2 causes a reduction 12-24 hours after treatment.

FIG. 25 demonstrates that FED and PAC decrease the viability of PC cells with higher efficacy than androgen withdrawal (ADT). Crystal violet staining and cell counting were used to compare PC viability over a time-course of 2-10 days between cells treated with ADT and control (+ADT), cells treated with PAC and control (+DMSO), cells treated with FED and control (+DMSO), cells treated with Jak2 knockdown (shJak2) and control (shCtrl), and cells treated with Stat5 knockdown (shStat5) and control (shCtrl).

FIG. 26 shows the results of a second study confirming the results shown in FIG. 25. Here, the effect of genetically knocking down AR (ShAR) was compared to control (shCtrl).

FIG. 27 demonstrates that overexpressing constitutively active (CA) Stat5 abolishes the PAC/FED-induced reduction in prostate cancer cell viability.

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The present inventor has discovered that Jak2-Stat5-signaling drives expression of full-length (FL) androgen receptor (AR) and its splice variants (AR-V), leading to increased AR protein levels in PC. In the Examples, this effect is demonstrated in PC cells in vitro, in tumors in vivo, and in patient-derived PC tissues cultured ex vivo. Importantly, the inventor found that blocking Jak2-Stat5 signaling reduces AR expression and inhibits tumor cell growth and proliferation. Thus, targeting Jak2 with specific Jak2 inhibitors that act through the Stat5 pathway (e.g., pacritinib, barocitinib, gandotinib, and fedratinib) can inhibit or reduce growth of androgen-sensitive prostate cancer and advanced prostate cancer cells (e.g., ENZ-resistant CRPC).

Jak2 tyrosine kinase phosphorylates the Stat5 signaling molecule in prostate cancer (PC), which leads to Stat5 dimerization and translocation to the nucleus to regulate transcription. Stat5 sustains PC cell viability and is critical for PC tumor growth in vivo. While the molecular targets downstream of Jak2-Stat5 signaling have been largely unclear, previous work has suggested that Stat5 may modulate the protein stability of AR in PC(6). The findings presented here indicate that Stat5 directly induces the transcription of the AR gene in PC.

Importantly, the inventor has discovered that this effect is limited to Jak2 inhibitors that act through the Stat5 pathway, as momelotinib (a Jak2 inhibitor that does not act through the Stat5 pathway) was not able to alter or reduce expression of full-length or variant AR in PC, nor was it able to reduce the growth of PC cells. This data demonstrates that not all Jak2 inhibitors are effective for the treatment of PC.

In 2009, a clinical trial of the the Jak2 inhibitor ruxolitinib for the treatment of PC patients was terminated early due to a lack of efficacy (“Study of Ruxolitinib (INCB018424) Administered Orally to Patients With Androgen Independent Metastatic Prostate Cancer”-ClinicalTrials.gov Identifier: NCT00638378). The failture of ruxolitinib signified to the pharmaceutical and medical fields that the pursuit of Jak2 inhibitors as treatments for PC was unlikely to succeed. Following this trial, the main focus of work in the PC field shifted to targeting Stat3 as opposed to Stat5(8). Thus, in this context, the observation that certain Jak2 inhibitors are highly effective inhibitors of PC cell growth and viability was unexpected and surprising.

The current clinical standard for treatment of advanced prostate cancer relies on a similar mechanism of antagonist action as older generation compounds (e.g., bicalutamide and flutamide), which inhibit the interaction between androgens and AR. For example, abiraterone (ABI, 17-(3-pyridyl)androsta-5,16-dien-3β-01) reduces the available ligands for the AR by blocking androgen biosynthesis. Likewise, luteinizing hormone-releasing hormone (LHRH) agonists (e.g., Lupron) indirectly inhibit androgen synthsis by the testis, thereby reducing circulating androgen levels (i.e., medical castration). Other drugs that act by similar mechanisms incluxw enzalutamide (ENZ), apalutamide APA, and darolutamide (DARO). Importantly, PCs that are resistant to LHRH agonists, ENZ, and ABI continue to express AR protein, typically at higher levels than in ENZ/ABI-naïve tumors. Additionally, AR-Vs are upregulated at this stage of the disease and function as constitutively active transcription factors that are impervious to actions of ENZ and ABI. The resistance to LHRH agonists or the newest generation of AR targeted-therapies is caused by persistent activities of these AR transcription factors. Thus, the inventor envisions that cancers resistant to therapeutics such as LHRH agonists, ENZ, ABI, APA, and DARO may be treated by the methods of the present invention.

Importantly, the methods of the present invention represent a substantial departure from the conventional therapeutic strategies of reducing circulating serum androgens or preventing the interaction of androgens with the AR ligand-binding domain (LBD). Specifically, the methods described herein employ a new strategy in which the expression of FL-AR and AR-Vs is eliminated by blocking Jak2-Stat5 signaling in androgen sensitive- and ENZ/ABI/APA/DARO-resistant CRPC using select Jak2 inhibitors, such as pacritinib (PAC) or fedratinib (FED).

As described in the Examples, the regulation of the AR by Jak2-Stat5 occurs at the transcriptional level in PC, and that Stat5 blockade almost entirely eliminates FL-AR and AR-V mRNA expression in both androgen-sensitive and CRPC cells to a level comparable to genetic knockdown of AR by lentiviral AR shRNA (shown as a control in each figure). Our data further show that pharmacological Jak2 inhibition by each of the new generation, selective Jak2 inhibitors (baricitinib, gandotinib, fedratinib and pacritinib) potently suppresses AR-FL and AR-V mRNA and protein expression in PC. While Jak2 inhibitors may also possess variable levels of Jak1 inhibitory activity, our data show that the Jak1-substrate, Stat3, does not regulate AR mRNA levels, demonstrating the specificity of the regulatory role of Stat5 on AR gene transcription.

The present invention relates to the use of pharmacological Jak2 inhibitors, for example, gandotinib, baricitinib, fedratinib, and pacritinib, to block growth of ENZ-resistant CRPC. Pacritinib (CTI BioPharma), a potent Jak2 inhibitor with a low toxicity profile, is currently in phase III trial for myelofibrosis. Fedratinib (Celgene/BMS) is under phase 3 trial in the treatment of myelofibrosis. No previous Jak2 inhibitor clinical trials have been conducted in the PC space for androgen-sensitive or ENZ-resistant active Stat5-positive PC.

Methods of inhibiting and reducing expression of androgen receptor (AR) and androgen receptor variants (AR variants) in both androgen-sensitive and CR prostate cancer cells are provided. The method comprises contacting the prostate cancer cell with an effective amount of a Jak2 inhibitor to inhibit or reduce AR and AR variant expression in the prostate cancer cell. The prostate cancer cell can be within a subject having androgen-sensitive organ-confined prostate cancer, androgen-sensitive advanced prostate cancer or CRPC.

The disclosure further provides a method of inhibiting or reducing growth of prostate cancer cells, including, androgen-sensitive prostate cancer cells (both organ confined and advanced prostate cancer cells) or castrate resistant prostate cancer (CRPC) and the method comprising administering a therapeutically effective amount of a Jak2 inhibitor that acts through Stat5 to inhibit or reduce growth of the PC cells within the subject. Not to be bound by any theory, but the provided method of inhibiting or reducing growth of the prostate cancer is believed to be due to the ability of the Jak2 inhibitor acting through the Stat5 pathway to directly act on the expression of AR, resulting in the reduction and inhibition the expression of androgen receptors (AR and AR variants) within the cancer cell, which in turn leads to cell cycle arrest, lack of proliferation and cancer cell death. The fact that momelotinib, a Jak2 inhibitor that fails to inhibit Stat5 activity, did not alter AR levels in PC cells supports the assertion that only a subset of Jak2 inhibitors have the necessary effect on Stat5 to reduce PC tumor cell growth.

The Jak2 inhibitors that can be used with the present invention are Jak2 inhibitors that affect the Stat5 signaling pathway. The present inventor discovered that certain Jak2 inhibitors are able to inhibit or reduce all AR expression (i.e., expression of both full-length AR and AR variants) within prostate cancer cells through the Stat5 pathway. This inhibition or reduction of AR expression within prostate cancer cells can be used to treat prostate cancer, including prostate cancer that is resistant to one or more anti-androgen therapies. Suitable Jak2 inhibitors that act through the Stat5 pathway and suppress AR levels include, for example, pacritinib, gandotinib, AZD1480, baricitinib, and fedratinib. These Jak2 inhibitors are commercially available, for example, pacritinib (available from Cell Therapeutics, IUPAC name (16E)-11-[2-(1-Pyrrolidinyl)ethoxy]-14,19-dioxa-5,7,26-triazatetracyclo[19.3.1.12,6.18,12]heptacosa-1(25),2(26),3,5,8,10,12(27),16,21,23-decaene), gandotinib (available from Eli Lily, IUPAC name 3-(4-Chloro-2-fluorobenzyl)-2-methyl-N-(5-methyl-1H-pyrazol-3-yl)-8-(morpholinomethyl)imidazo[1,2-b]pyridazin-6-amine), AZD1480 (available from AstraZeneca, IUPAC name 5-chloro-2-N-[(1S)-1-(5-fluoropyrimidin-2-yl)ethyl]-4-N-(5-methyl-1H-pyrazol-3-yl)pyrimidine-2,4-diamine, described in US20180263995A1), baricitinib (available from Eli Lilly, IUPAC name 2-[1-Ethylsulfonyl-3-[4-(7H-pyrrolo[2,3-d]pyrimidin-4-yl)pyrazol-1-yl]azetidin-3-yl]acetonitrile), and fedratinib (available from BMS/Celgene IUPAC name N-tert-Butyl-3-{5-methyl-2-[4-(2-pyrrolidin-1-yl-ethoxy)-phenylamino]-pyrimidin-4-ylamino}-benzenesulfonamide). In a preferred embodiment, the Jak2 inhibitor is pacritinib or fedratinib.

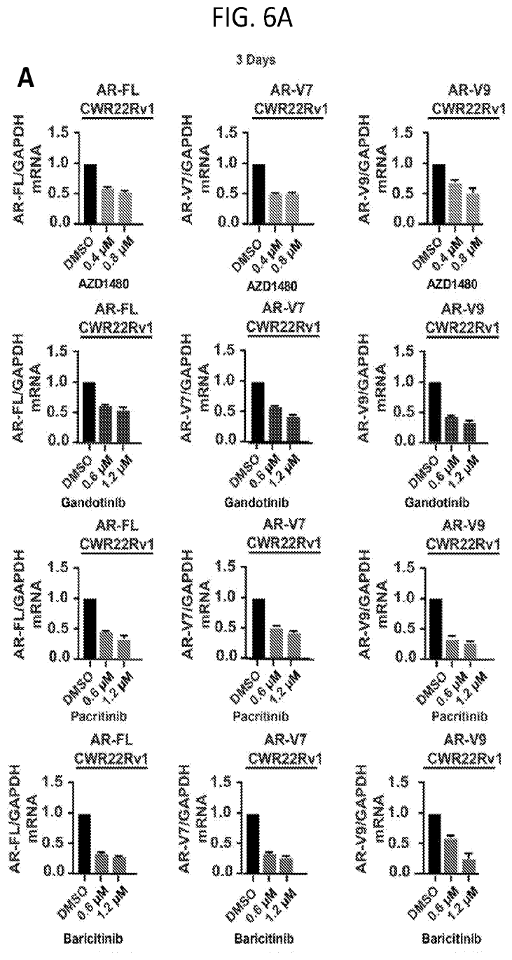

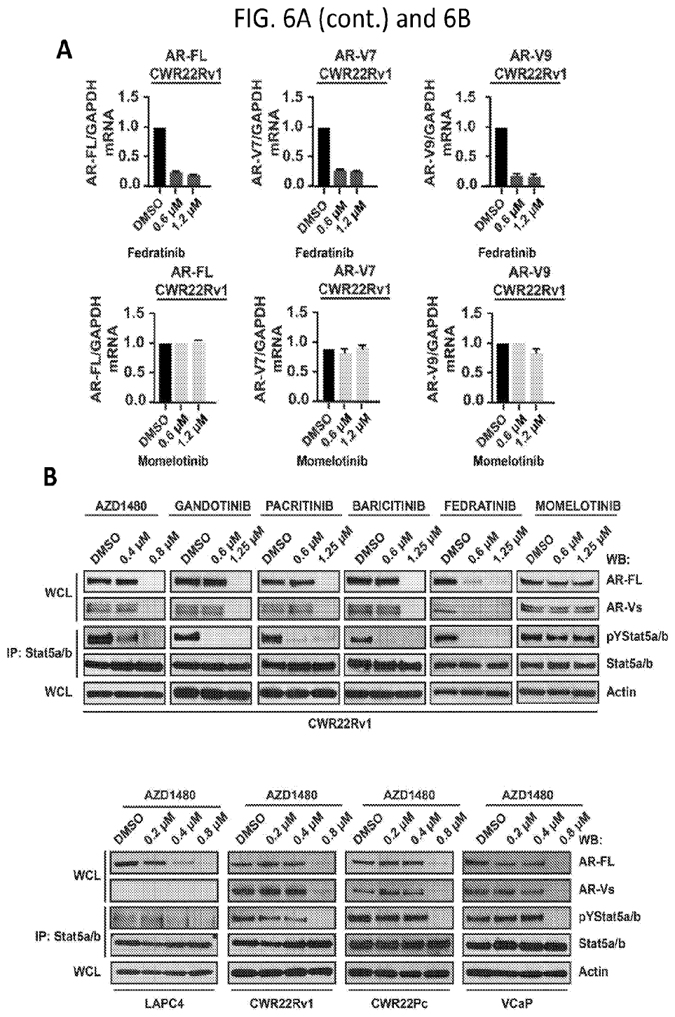

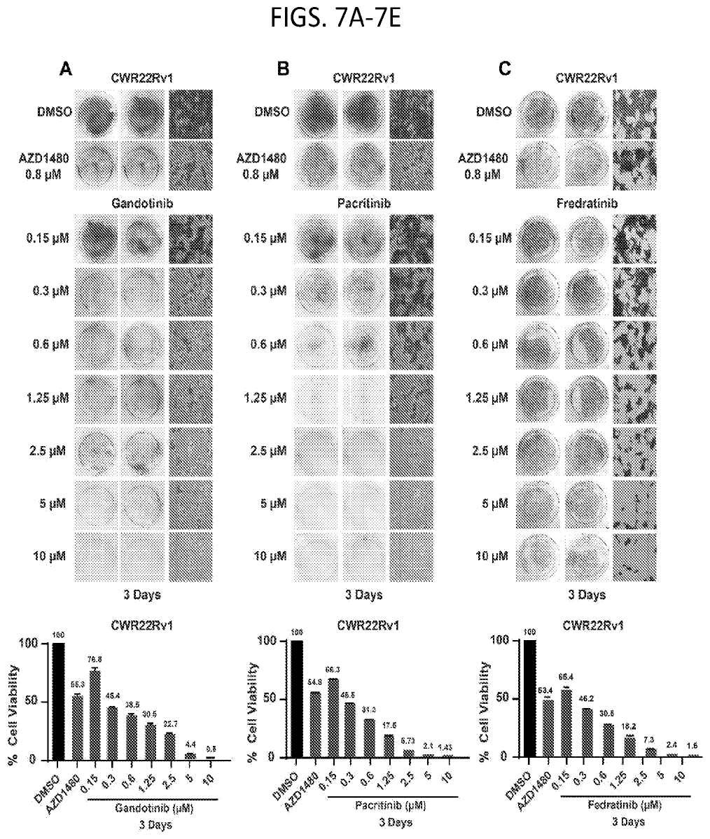

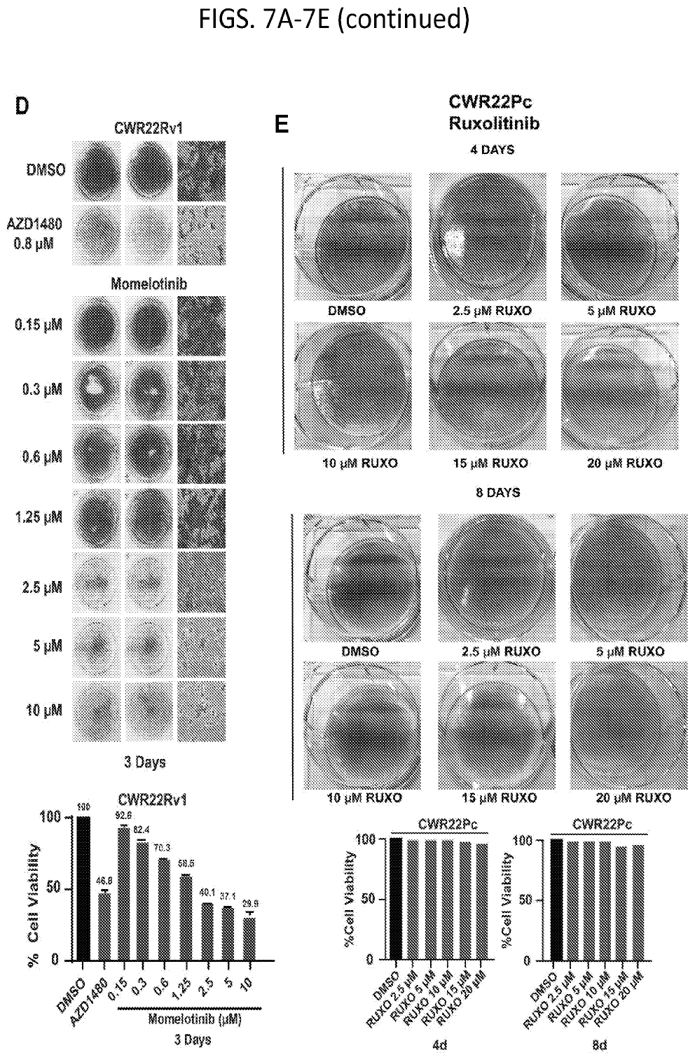

As is demonstrated in FIG. 7D, not all Jak2 inhibitors are able to inhibit the growth of prostate cancer cells, particularly castrate resistant prostate cancer cells. Momelotinib, a Jak2 inhibitor that does not inhibit Stat5 phosphorylation, does not affect the AR levels in prostate cancer cells and does not reduce PC cell viability (see FIGS. 6B and 7D). Thus, the Jak2 inhibitors of the present invention work specifically through the Jak2/Stat5 pathway, and Jak2 inhibitors that do not affect Stat5 signalling would not work in the methods of the present invention.

The methods described herein can be used to treat androgen-sensitive prostate cancer, including both androgen-sensitive organ-confined prostate cancer, androgen-sensitive advanced prostate cancer and castrate resistant prostate cancer (CRPC). CRPC continues to grow even when the amount of testosterone in the body is reduced to very low levels while early-stage prostate cancers need normal levels of testosterone to grow. CRPC is a form of prostate cancer that is not responding to first-line androgen deprivation therapy or treatment with the conventional androgen deprivation (LHRH agonists or antagonists) or androgen receptor antagonists such as anti-androgens.

More specifically, the methods described herein can be used on prostate cancer or CRPC that is resistant to treatment by one or more anti-androgen therapies, including medical castration. In one embodiment, the PC or PRPC is resistant to treatment with a luteinizing hormone-releasing hormone (LHRH) agonist, enzalutamide, apalutamide, darolutamide or abiraterone.

Enzalutamide or ENZ (commercially available from Astellas Pharma, Northbrook Ill., IUPAC name: 4-(3-(4-cyano-3-(trifluoromethyl)phenyl)-5,5-dimethyl-4-oxo-2-thioxoimidazolidin-1-yl)-2-fluoro-N-methylbenzamide) is a nonsteroid antiandrogen (NSAA) used in treatment of prostate cancer and specifically CRPC, both metastatic castration-resistant prostate cancer (mCRPC) and nonmetastatic castration-resistant prostate cancer. In some embodiments, the methods described herein are for the reduction or inhibition of AR or AR variants in ENZ/ABI/APA/DARO-resistant prostate cancer cells. Anti-androgen resistant prostate cancer, includes, ENZ-resistant, ABI-resistant, APA-resistant, DARO-resistant prostate cancer, among others. By “ENZ-resistant” prostate cancer we mean prostate cancer cells that are not inhibited in growth by treatment with ENZ, by “ABI-resistant” prostate cancer we mean prostate cancer cells that are not inhibited in growth by treatment with ABI, by “APA-resistant prostate cancer we mean prostate cancer cells that are not inhibited in growth by treatment with APA, by “DARO-resistant” prostate cancer we mean prostate cancer cells that are not inhibited in growth by treatment with DARO. Other anti-androgen resistant prostate cancers are contemplated to be treated by the methods described herein.

Abiraterone or ABI (available from Janssen Biotech (commercial name Zytiga®), IUPAC name [(3S,8R,9S,10R,13S,14S)-10,13-dimethyl-17-pyridin-3-yl-2,3,4,7,8,9,11,12,14,15-decahydro-1H-cyclopenta[a]phenanthren-3-yl] acetate) is an antiandrogen therapy that blocks cytochrome p17 and inhibits the production of androgens like testosterone and dihydrotestosterone in the body. By the term abiraterone used herein, the prodrug, abiraterone acetate, is also contemplated. The prostate cancer treated herein may be resistant to ABI treatment (i.e., ABI-resistant cancer).

Apalutamide (APA, Erleada®, 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide, Janssen) is a nonsteroidal antiandrogen medication and used for treatment of non-metastatic castration-resistant prostate cancer.

Darolutamide (DARO, N—((S)-1-(3-(3-Chloro-4-cyanophenyl)-1H-pyrazol-1-yl)propan-2-yl)-5-(1-hydroxyethyl)-1H-pyrazole-3-carboxamide, Bayer) is another nonsteroidal antiandrogen that is a selective antagonist of AR for treatment of advanced, castration-resistant prostate cancer.

“Medical castration” or “chemical castration” is refers to the treatment of a prostate cancer patient with hormone therapy, e.g., androgen deprivation therapy, that prevents secretion of hormones from the testis, and include, but are not limited to, for example, luteinizing hormone-releasing hormone (LHRH) agonists, which prevent secretion of luteinizing hormone, LHRH agonists or LHRH analogs, gonadotropinb-releasing hormone (GnRH) antagonists, including, for example, leuprorelin and Goserelin, among others. Chemical castration compounds are including as androgen deprivation therapy as described herein.

As used herein, “treatment” or “treating” refers to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. Specifically, treatment results in the reduction in tumor load or volume in the patient, and in some instances, leads to regression and elimination of the tumor or tumor cells, including a reduction in the number of tumor cells. As used herein, the term “treatment” is not necessarily meant to imply cure or complete abolition of the tumor. Treatment may refer to the inhibiting or slowing of the progression of the tumor, reducing the incidence of tumor, reducing the size of a tumor, reducing the number of tumor cells within a subject, reducing metastasis of the tumor, reducing the surrogate of the tumor or metastases growth in serum such as PSA, reducing radiographically tumor metastases growth or preventing additional tumor growth or development of metastases. In some embodiments, treatment results in complete regression of the tumor.

By “ameliorate,” “amelioration,” “improvement” or the like we mean a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated with the compositions of the present invention, where the untreated subjects have, or are subject to developing, the same or similar tumor.

As is known in the art, a “cancer” or “tumor” is generally considered as uncontrolled cell growth. The terms “cancer” and “tumor” are used herein interchangeably. The methods described herein may be used to treat a cancer which have an upregulation of androgen receptor (AR) associated with the cancer, for example, prostate cancer, more specifically both androgen-sensitive and castrate-resistant prostate cancer, including cancers resistant to one or more forms of anti-androgen therapy.

The term “effective amount” or “therapeutically effective amount” refers to an amount sufficient to effect beneficial or desirable biological and/or clinical results or stabilization of the disease.

It will be appreciated that appropriate dosages of the Jak2 inhibitor, and compositions comprising a Jak2 inhibitor, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, or materials used in combination, and the age, sex, weight, condition, general health, and prior medical history of the patient. The amount of compound and route of administration will ultimately be at the discretion of the physician. Administration in vivo can be effected in one dose, continuously or intermittently (e.g. in divided doses at appropriate intervals) throughout the course of treatment.

Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that is suffering from cancer (e.g., prostate cancer). In one embodiment, the subject is suffering from androgen-sensitive prostate cancer, either organ-confined or advanced. In a preferred embodiment, the patient is suffering from an aggressive form of the cancer, for example an aggressive form of prostate cancer. In some embodiments, the patient is suffering from castrate-resistant prostate cancer. In some embodiment, the patient is suffering from locally advanced prostate cancer or disseminated prostate cancer that has metastasized to other organs. In some embodiments, the patient is a subject that has prostate cancer that is or has become resistant to anti-androgen therapy, for example, the prostate cancer is resistant to a luteinizing hormone-releasing hormone (LHRH) agonist; enzalutamide, ENZ; apalutamide, APA; darolutamide, DARO; or abiraterone, ABI.

In some embodiments, the subject is further administered an anti-androgen therapy in addition to the Jak2 inhibitor that acts through Stat5 described herein. For example, the androgen deprivation therapy can include anti-androgen therapy known in the art, including, for example, enzalutamide; apalutamide, darolutamide; or abiraterone, or medical castration (LHRH agonist/GNRH antagonist), among others.

In a preferred embodiment, the Jak2 inhibitor is fedratinib, pacritinib, baricitinib or gandotinib.

In another embodiment, a method of inhibiting, reducing or eliminating ENZ-resistant CRPC cell growth or anti-androgen-resistant cell growth in a subject with prostate cancer is contemplated. The method comprising administering an effective amount of a Jak2 inhibitor and an effective amount of ENZ, APA, DARO or ABI to the subject with prostate cancer in order to treat the cancer and inhibit growth of ENZ/APA/DARO/ABI-resistant prostate cancer cells within the subject. By “inhibiting ENZ-resistant CRPC cell growth” there is a reduction or elimination in the growth of prostate cancer cells that are resistant to treatment from ENZ or other anti-androgen therapies. In some embodiments, the growth of ENZ/APA/DARO/ABI-resistant CRPC is reduced by at least about 25%, more preferably at least about 30%, alternatively at least 40%, at least about 50%, at least about 60%, at least about 70%, at least about 75%, at least about 80%, at least about 85%, at least about 90% reduction in the growth of ENZ/APA/DARO/ABI-resistant CRPC cells. In another embodiment, the “reduction or elimination of growth of prostate cancer cells” includes disease stabilization, i.e., limiting new cancer cell growth within the subject. In a preferred embodiment, the Jak2 inhibitor is pacritinib, fedratinib, baricitinib or gandotinib.

In some embodiments, a method of inhibiting, reducing or eliminating ABI-resistant CRPC, DARO-resistant CRPC, or APA-resistant cell growth is provided. The method comprises administering an effective amount of Jak2 inhibitor in order to reduce, inhibit or eliminate the growth of ABI-resistant DARO-resistant CRPC, or APA-resistant CRPC cells. In some embodiments, the Jak2 inhibitor is administered in combination with an effective amount of ABI, DARO, or APA in order to treat the cancer along with inhibiting, reducing or eliminating ABI-resistant, DARO-resistant CRPC, or APA-resistant CRPC cell growth.

By “administering” we mean any means for introducing the Jak2 or Stat5 inhibitor described herein into the body, preferably orally or into the systemic circulation. Examples include but are not limited to oral, buccal, sublingual, pulmonary, transdermal, transmucosal, as well as subcutaneous, intraperitoneal, intravenous, and intramuscular injection. A preferred method of administration is orally.

In some embodiments, the method further comprises administering to the subject a second cancer therapy. The combination of the Jak2 or Stat5 inhibitor and the second cancer therapy results in an increase in the efficacy of the treatment of the cancer than the second therapy administered alone and includes a reduction in the development of castrate resistant prostate cancer. Suitable second cancer therapies are known in the art and include, but are not limited to, for example, chemotherapy, radiation, surgery, vaccine therapy and combinations thereof. In a preferred embodiment, the secondary cancer therapy is an androgen deprivation therapy (androgen deprivation therapy can be carried out by multiple ways—including anti-androgens or medical castration), for example, an antiandrogen therapy.

The use herein of the terms “including,” “comprising,” or “having,” and variations thereof, is meant to encompass the elements listed thereafter and equivalents thereof as well as additional elements. Embodiments recited as “including,” “comprising” or “having” certain elements are also contemplated as “consisting essentially of” and “consisting of” those certain elements.

The present invention has been described in terms of one or more preferred embodiments, and it should be appreciated that many equivalents, alternatives, variations, and modifications, aside from those expressly stated, are possible and within the scope of the invention.

### Example 1—Aberrant Androgen Receptor Expression in Prostate Cancer

This example demonstrates the expression of full length and variants of androgen receptor (AR) in prostate cancer and the ability of Jak2 inhibitors that act through the Stat5 signaling pathway to inhibit PC cell growth. The main protein that drives disease progression in PC is the AR, a transcription factor which is induced by androgenic steroids (e.g. testosterone) to regulate many of the genes that support tumor growth(9). The AR, a member of the nuclear receptor family, contains an N-terminal transcriptional activation domain, a central DNA-binding domain and C-terminal ligand-binding domain(10, 11). Steroid binding to the ligand-binding domain signals nuclear localization followed by chromatin engagement of two monomers of AR as a dimer at androgen response elements of target genes(10-12). Currently used AR antagonists, such as ENZ, block AR activation by competitive inhibition of steroid binding to the androgen-binding pocket on the ligand-binding domain(3). ABI is a potent androgen synthesis inhibitor, which provides systemic depletion of testosterone and a systemic blockade of AR activation(4). APA, a selective competitive silent antagonist of AR, and darolutamide (DARO) an androgen receptor antagonist are also used for treatment of PC, including CRPC. Androgen deprivation can also be carried out by inhibition of testosterone synthesis and secretion by LHRH agonists or GNRH antagonists to suppress LH hormone resulting in suppression of serum testosterone levels. These can be combined with the Jak2 inhibitors of the present invention to treat prostate cancer.

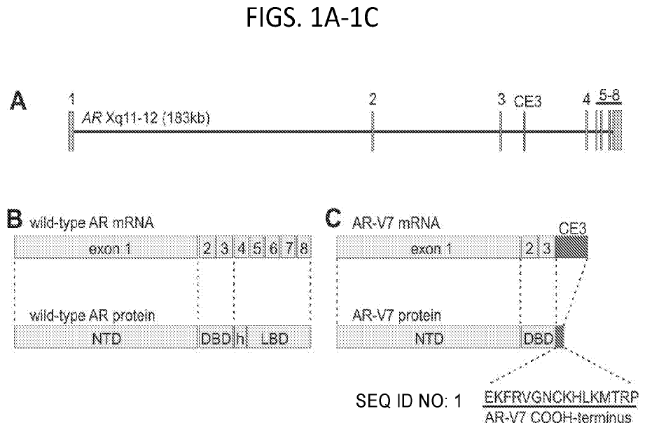

Resistance to these AR-targeted therapies is multi-factorial, but prior work has shown that the vast majority of CRPC tumors harbor heritable, DNA-level changes in the AR gene. The most frequent alteration in the AR gene is amplification, which occurs in 60-65% of CRPC and drives AR protein overexpression(13, 14). AR protein overexpression sensitizes tumors to castrate levels of androgens(13). Additionally, AR gene amplification promotes overexpression of AR-V7, a splicing variant of AR that lacks the ligand-binding domain and displays constitutive transcriptional activity (FIG. 1)(15). We have shown that AR-V7 is frequently co-expressed with additional AR splicing variants that have been identified in CRPC, including AR-V1 and AR-V9(16, 17). Thus, in conclusion, prostate cancer is associated with overexpressed, mutated, and truncated AR proteins in CRPC tumors.

### Example 2—the Jak2-Stat5 Axis Drives Androgen Receptor Expression in Prostate Cancer

This Example provides new data on an entirely novel concept that Stat5 drives the expression of all of the aberrant forms of the AR in PC, and the Jak2-Stat5 axis represents a target to inhibit their expression in CRPC and thereby control ENZ/ABI-resistant CRPC growth.

Jak2 tyrosine kinase(18, 19) is a member of Jak family including Jak1, Jak3 and Tyk2. Jak2 has seven Jak homology (JH) domains where 1111 domain represents the kinase domain, JH2 the pseudokinase domain, the JH3-JH4 domains share homology with SH2-domains, and JH5-JH7 domain represents the FERM domain(20, 21). The binding of cytokines, hormones and growth factors to their specific receptors results in multimerization with cytoplasmic domains that are associated with Jak2. This conformational change results in autophosphorylation and activation of the Jak2 protein where Jak2 catalyzes transfer of phosphate from the ATP molecule to its own tyrosine residues and cytoplasmic signaling proteins to activate them. Amino acid residues Tyr1007/1008 in the activation loop of the JH1 are required for full catalytic activity(20, 21). The pseudokinase domain in Jak2 is catalytically inactive but is crucial in providing negative inhibition for the basal Jak2 kinase activity. Jak2 is the key kinase phosphorylating Stat5 in PC(19, 22-24). Stat5 comprises two highly homologous isoforms Stat5a (94 kDa) and Stat5b (92 kDa) (referred to as Stat5) which are nucleocytoplasmic proteins acting both as signaling proteins and nuclear transcription factors(25-29). Upon tyrosine phosphorylation, Stat5 forms functional dimers that translocate to the nucleus and bind to specific Stat5 DNA response elements(27). Numerous Jak2 inhibitors have been developed over the past 10 years, such as AZD1480, CHZ868, BMS911543, ruxolitinib, fedratinib, momelotinib, XL019, GLPG0634, baricitinb, and gandotinib. Generally, the key issue in the clinical development of Jak2 inhibitors has been neurotoxicity due to off-target effects on Trk kinases in brain tissue (a problem that is particularly relevant for AZD1480). The new generation Jak2 inhibitors, pacritinib (PAC)(30-34) and fedratinib both have low toxicity profile and are currently in phase III clinical trials for the treatment of myelofibrosis.

Transcription Factor Stat5 and PC Growth:

Stat5 sustains PC cell viability and induces PC growth(35)(24, 36-39). Blockade of Stat5 signaling induces apoptotic death of PC cells, suppresses growth of both xenografted and autochthonous PC tumors as well as clinical patient-derived PCs ex vivo in culture(24, 36, 38-43). Conversely, overexpression of active Stat5 induces proliferation of PC cells in culture and growth of PC tumors in mice(44). In 30-40% of advanced CRPCs, the chromosome 17 locus encompassing STAT5A and STAT5B genes undergoes amplification by cytological analysis resulting in increased Stat5 protein levels(44). Notably, Stat5 activation in PC predicts early PC recurrence(45, 46). The mechanisms underlying Stat5 sustenance of PC cell viability have been largely unclear.

Stat5 has been shown to modulate the protein stability of AR in PC(6, 43). However, the observed effect was moderate and could not account for the biological effects of Stat5 in sustaining PC cell viability. This Example demonstrates that Jak2 drives FL-AR and AR-V mRNA expression in PC via Stat5 (but not Stat3). This suggests that targeting Jak2 with pacritinib or another Jak2 inhibitor with similar features represents a new therapeutic strategy to eliminate FL-AR and AR-Vs in ENZ-resistant CRPC and block CRPC growth.

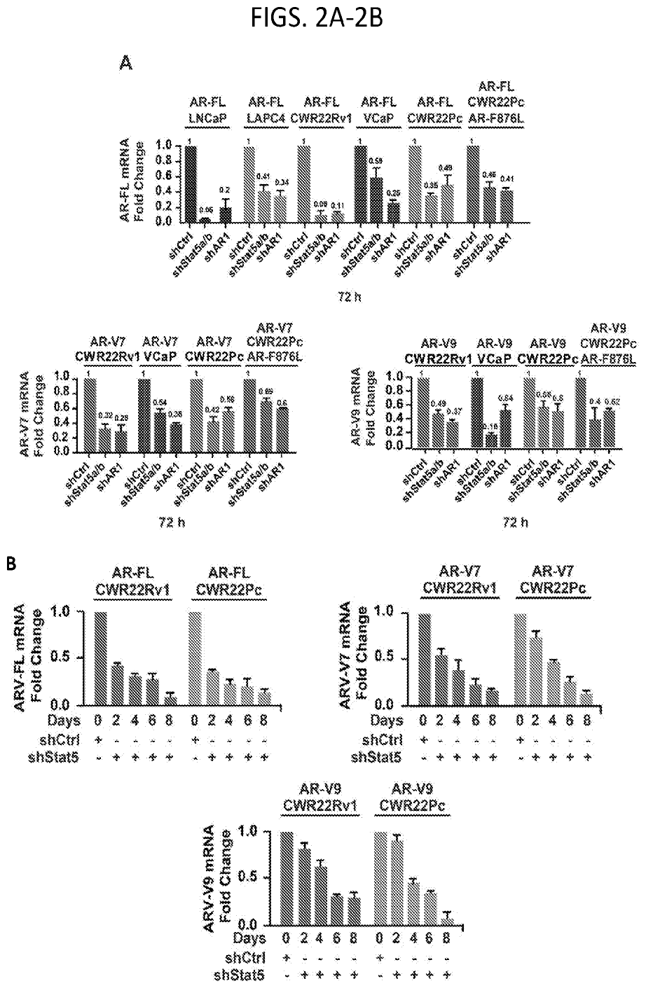

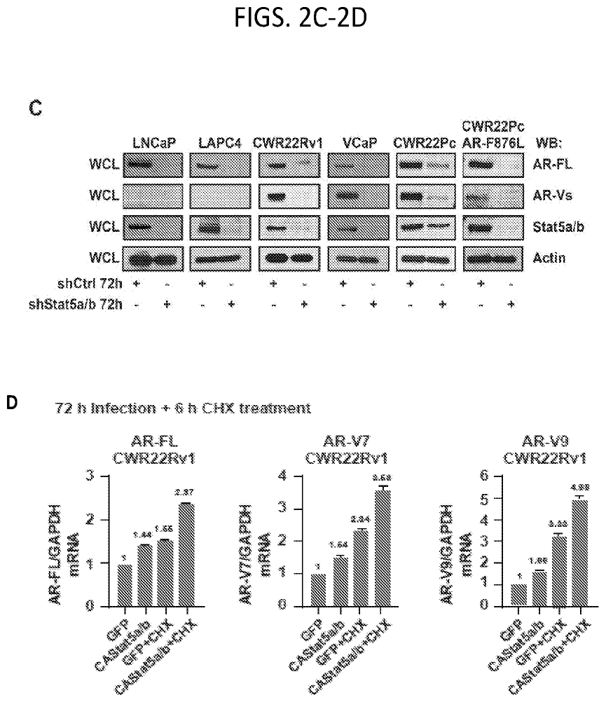

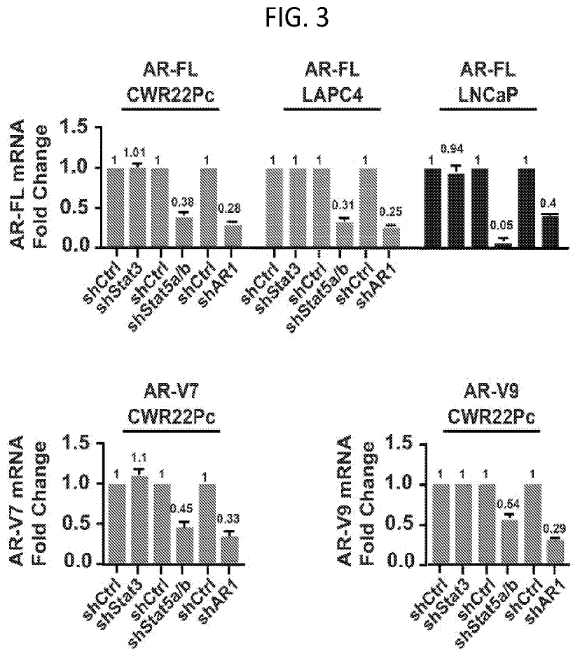

Inhibition of Stat5 suppresses AR (FL/AR-Vs) mRNA and protein expression in PC, while constitutive active Stat5 increases AR (FL/AR-Vs) mRNA and protein expression in PC: To investigate whether AR is a Stat5 target gene in PC, Stat5 was inhibited in a panel of PC cell lines by lentiviral expression of Stat5a/b targeting shRNA (Stat5 shRNA) and the mRNA levels of FL-AR, AR-V7 and AR-V9 were evaluated by quantitative RT-PCR. Stat5 knockdown down-regulated mRNA levels of both FL-AR and the AR splice variants AR-V-7 and AR-V9. Of note, the degree of FL-AR and AR-V inhibition achieved by Stat5 knock-down was the same as directly knocking down AR with AR shRNA in all PC cell lines tested (FIG. 2A). In fact, Stat5 knockdown reduced the mRNA levels of FL-AR, AR-V7 and AR-V9 by 80-90% 8 days after lentiviral Stat5 shRNA expression, shown in FIG. 2B. Stat5a/b was inhibited by lentiviral expression of Stat5 shRNA in PC cell lines. FIG. 2C shows that genetic depletion of Stat5a/b resulted in reduced AR protein expression in all PC cells lines tested. Importantly, knock-down of Stat3 did not affect AR mRNA levels in any of the cell lines, indicating that this effect is specific to Stat5 (FIG. 3).

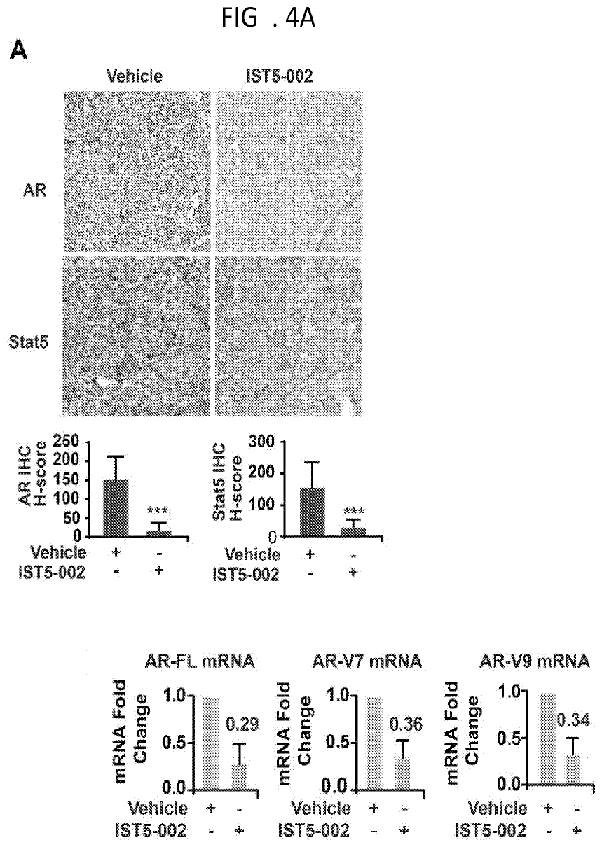

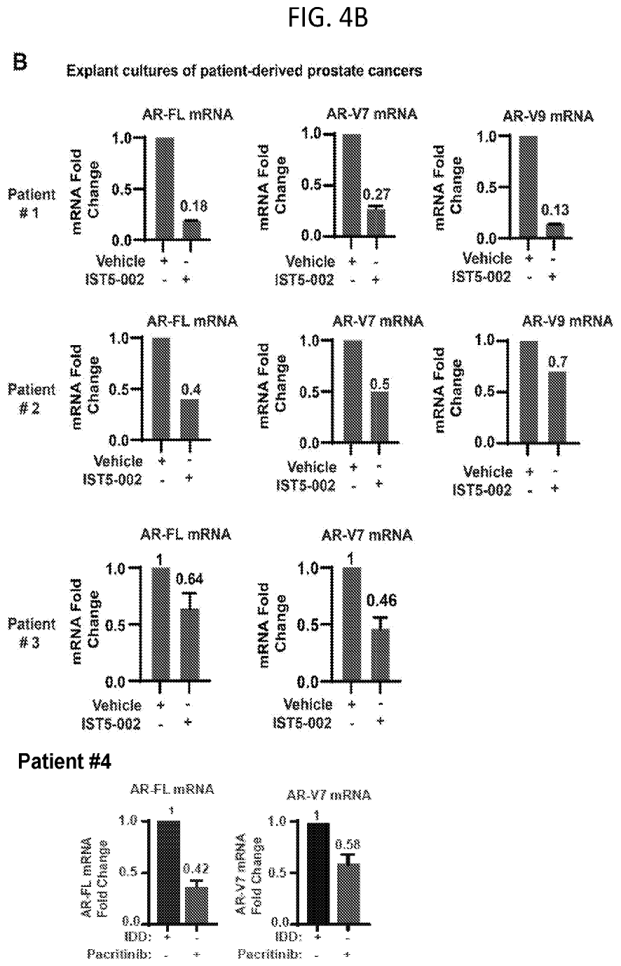

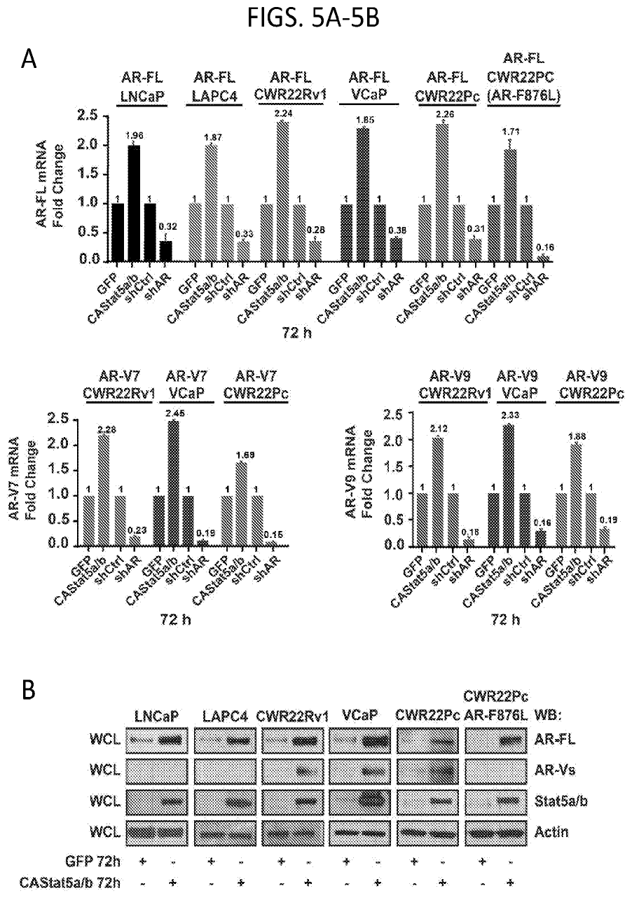

To evaluate whether Stat5 regulation of AR mRNA and protein levels is evident in an in vivo setting where PC cells were grown as xenograft tumors in mice, CWR22Pc cells (1.5×10⁷) were inoculated subcutaneously into flanks of nude mice (one tumor/mouse). After tumors reached approximately 100 mm³ in size (12 d), mice were treated daily for 40 days by intraperitoneal injection with IST5-002 (40 mg/kg body weight) or vehicle (0.3% hydroxypropyl cellulose/H₂O) (5 mice/group). AR mRNA and protein levels were evaluated by q-RT-PCR(47) and by immunohistochemistry, as previously described(18, 45, 46, 48, 49). As shown in FIG. 4A, suppression of Stat5 by IST5-002 resulted in a marked decrease in AR mRNA and protein expression in PC tumors in vivo.

The finding of Stat5 regulating AR mRNA was recapitulated in patient-derived PC tissues cultured ex vivo in 3D tumor explant cultures for 7 days in the presence or absence of PAC or IST5-002. As shown in FIG. 4B, both full-length AR and AR-V7 mRNA levels were decreased in clinical PCs ex vivo by 40-50% by Stat5 inhibition in three individual patients. These data indicate that Jak2-Stat5 signaling is critical for FL-AR and AR-V7 mRNA expression not only in cell cultures models of PC but also in PC xenograft tumors and in clinical patient-derived PCs.

Constitutively active mutants of Stat5a/b (CAStat5a/b; Stat5aS710F, Stat5bS715F)(24) were lentivirally expressed in a panel of PC cell lines, and mRNA levels of FL-AR and AR splice variants (AR-V7, AR-V9) were determined by quantitative RT-PCR. Expression of CAStat5a/b resulted in a uniform increase in mRNA levels of FL-AR, AR-V7 and AR-V9 in all PC cell lines tested (FIG. 5A). Furthermore, expression of CAStat5a/b resulted in a robust increase in AR protein expression in all PC cell lines tested, shown by immunoblotting (FIG. 5B). In summary, our data show that active Stat5 sustains FL-AR and AR-V mRNA levels in PC resulting in increased AR protein expression.

To determine if Stat5 is a direct transcriptional regulator of the AR gene, active Stat5 (CAStat5) was expressed via lentivirus for 72 h followed by cyclohexamide (CHX) treatment for 24 h. Inhibition of new protein synthesis by CHX did not block the induction of AR transcription by active Stat5 (CAStat5).

Jak2-Inhibitors Suppress Stat5 Signaling and AR Levels in PC Cells:

We tested whether Stat5 regulation of AR mRNA and protein levels provides an opportunity to control AR levels pharmacologically by inhibition of Jak2 signaling. Jak2 activity was inhibited using the Jak2 inhibitors AZD1480, gandotinib, pacritinib (PAC), baricitinib, fedratinib, and momelotinib. These Jak2 inhibitors caused a dose-dependent reduction in both FL and AR-V AR mRNA and protein levels in CWR22Rv1 CRPC cell line (FIGS. 6A-6B). Finally, to evaluate the effect of PAC and FED on PC cell viability, CWR22Rv1 cells were treated with increasing concentrations of PAC or FED for 3 days. At 300 nM, more than 50% of the cells were eliminated by both PAC and FED (FIGS. 7A-7D). Further, FIG. 7D demonstrates that a Jak2 inhibitor (i.e., momelotinib) that acts through a different pathway from Stat5 does not alter AR expression or result in an increased PC cell death as with the Jak2 inhibitors that modulate AR through Stat5. In conclusion, these results demonstrate that inhibition of Jak2-Stat5 signaling leads to depletion of FL-AR and AR-V mRNA and protein expression in PC cells in vitro, in PC tumors in vivo and in patient-derived PCs tested ex vivo. Moreover, pacritinib, gandotinib and fedratinib induce death of PC cells in vitro.

### Example 3—Enzalutamide (ENZ)-Liganded Androgen Receptor Induces Jak2-Stat5 Signaling in Prostate Cancer

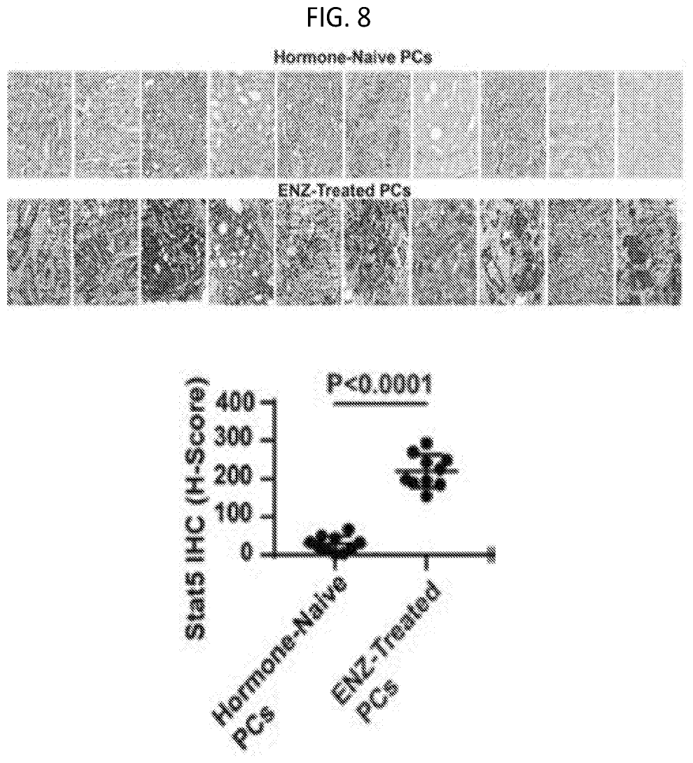

We recently reported that ENZ induces a hyperactivated feed-forward Jak2-Stat5 signaling loop in PC to promote ENZ-resistant PC growth(50). Immunohistochemical analyses of the activation status of Stat5 in PCs of patients treated with ENZ show that the levels of nuclear active Stat5 were significantly (p<0.0001) elevated in biopsies of ENZ-treated PCs when compared to hormone-naïve PCs of corresponding histological grades (FIG. 8).

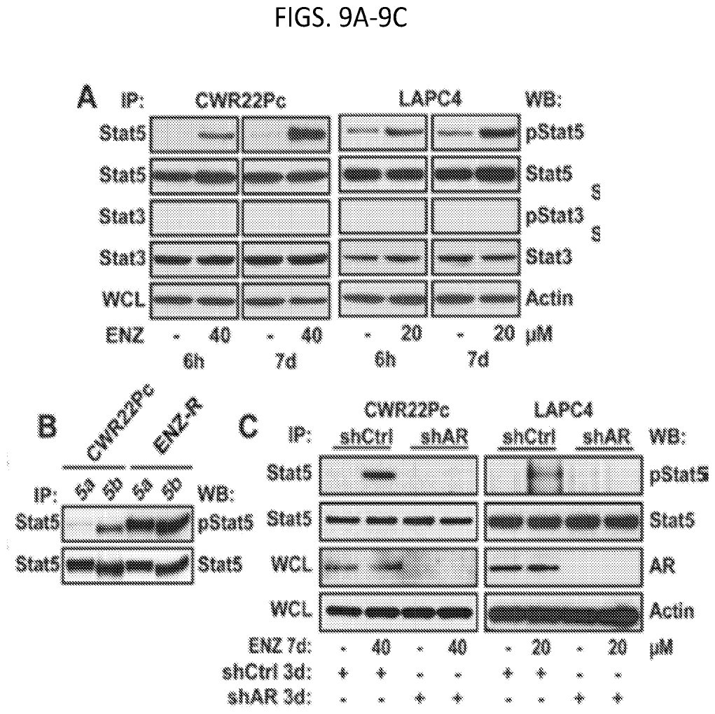

Also, in paired PC samples from patients before and after ENZ treatment (average 6 mo), active Stat5 levels were robustly elevated in the biopsies after ENZ treatment compared to the biopsies taken prior to ENZ treatment(50). Similar to the clinical PC samples, ENZ induced a robust Stat5 phosphorylation in PC cell lines which was sustained (FIG. 9). In a CWR22Pc cell subline expressing AR-F876L (CW22Pc-ENZ-R)65, which emerges in CWR22Pc cells surviving extended treatment with ENZ (>6 months), active Stat5 levels were markedly higher compared to the parental CWR22Pc cells (FIG. 9B). To investigate whether AR is required for ENZ induction of Stat5 signaling, we inhibited AR expression in CWR22Pc and LAPC4 cells by genetic knockdown using lentiviral expression of AR shRNA (FIG. 9C). In the absence of AR, ENZ failed to induce Stat5 activation demonstrating that ENZ-induction of Stat5 signaling is not an AR-independent off-target effect of the ENZ compound. Of note, androgen deprivation, additional androgen treatment or genetic knockdown of AR did not increase Stat5 phosphorylation in PC cells(50).

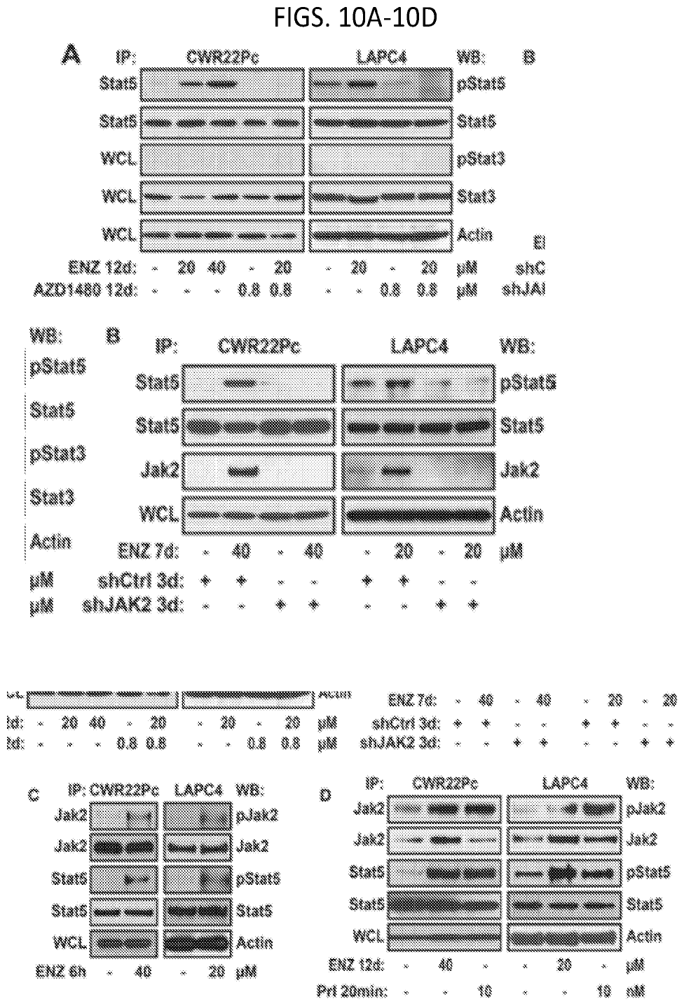

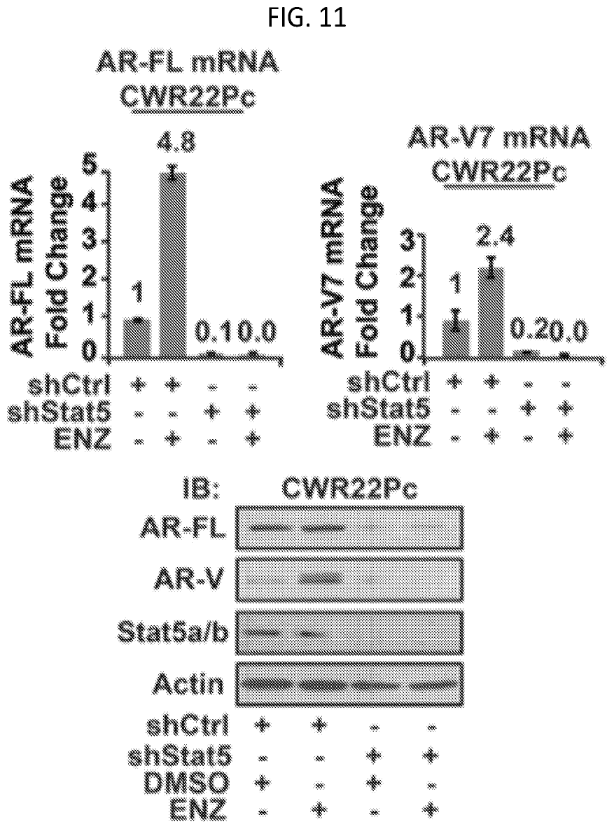

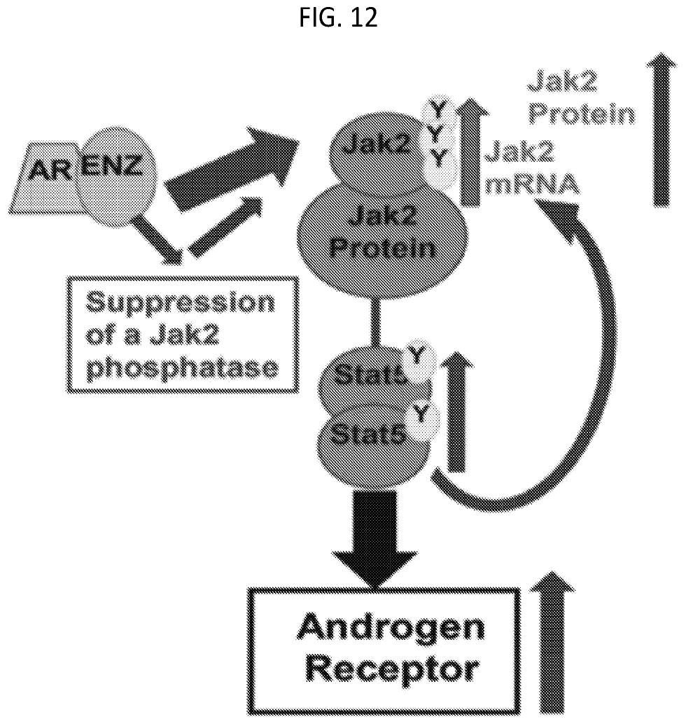

To assess if ENZ-induced Stat5 activation in PC cells is dependent on Jak2 signaling, Jak2 activity was pharmacologically inhibited by Jak2-inhibitor AZD1480 during ENZ-treatment. AZD1480 blocked ENZ-induced Stat5 phosphorylation in CWR22Pc, LAPC4 and CWR22Pc-ENZ-R cells (FIG. 10A). To further assess the involvement of Jak2 in ENZ-induced Stat5 activation, Jak2 was genetically knocked down by lentiviral expression of Jak2 shRNA in CWR22Pc and LAPC4 cells for 3 days followed by ENZ treatment for 7 days. In the absence of Jak2, ENZ failed to induce Stat5 phosphorylation in both cell lines (FIG. 10B). At the same time, our data show that ENZ induced robust phosphorylation of Jak2 in PC cells which was sustained and further increased (FIGS. 10C-10D). Mechanistically, our data show that ENZ-liganded AR induces rapid and sustained Jak2 phosphorylation in PC cells through a process involving Jak2-regulatory protein SHP2. Here, we further show that ENZ-induction of Jak2-Stat5 signaling leads to induction of the AR levels in PC (FIG. 11). In conclusion, ENZ-induced Jak2 activation lead to a formation of a feed-forward loop in PC cells where active Stat5 increases Jak2 mRNA and protein levels 1. Hyperactivated Jak2-Stat5 signaling loop induces the expression and levels of the AR in PC cells (FIG. 12). Previously, we demonstrated that inhibition of Stat5 as a second-line treatment induced death of PC cells surviving ENZ treatment. Specifically, we demonstrated that activated Stat5 promoted growth of PC cells during ENZ treatment and, at the same time, inhibition of Stat5 as a second-line treatment induced extensive death of PC cells surviving ENZ treatment. Pharmacological Jak2-Stat5 blockade inhibited CR growth of PC xenograft tumors after ENZ resistance developed and induced further death after ENZ treatment in patient-derived PCs ex vivo in tumor explant cultures. In summary, this work indicates a pivotal role of hyperactivated Jak2-Stat5 signaling in ENZ-resistant PC which is readily targetable by Jak2 inhibitors in clinical development.

Next, we evaluated whether inhibition of Stat5 as a second-line treatment induces extensive death of PC cells surviving ENZ treatment. Stat5 blockade inhibited CR growth of PC xenograft tumors after ENZ resistance developed and induced further death after ENZ treatment in patient-derived PCs ex vivo in tumor explant cultures(50). In summary, this work introduces a novel concept of a pivotal role of hyperactivated Jak2-Stat5 signaling in ENZ-resistant PC which is readily targetable by Jak2 inhibitors in clinical development.

### Example 4—ENZ Induces De-Differentiation of PC Cells, Metastatic PC Cell Phenotype and Metastatic Progression of PC, which is Mediated by Jak2-Stat5 Signaling

Our previous work demonstrated that Stat5 induces metastatic dissemination of PC, as evidenced by Stat5 promotion of formation of distant metastases of PC in vivo, and induction of hallmarks of EMT and stem-like cancer cell properties through induction of Twist1 and BMI1 expression in PC(24). Specifically, Stat5 induced a substantial increase in N-cadherin and other markers of EMT such as Vimentin, Fibronectin and Twist1 not only in PC cell lines, but also in PC xenograft tumors and in patient-derived PCs ex vivo. Stat5 induction of EMT in PC was blocked by genetic or pharmacological inhibition of Jak2. In addition to promotion of phenotypic markers of EMT in PC, Stat5 induced functional endpoints of metastatic behavior of PC cells. Specifically, Stat5 increased migration of PC cells in Boyden chamber assays, disrupted growth of PC cells as epithelial monolayers and increased anchorage-independent survival of small PC cell clusters. Also, Stat5 triggered an emergence of pre-metastatic niches as demonstrated by increased adhesion of PC cells to Fibronectin. Twist1 was identified as the key mediator of the Stat5-induced EMT in PC. At the same time with induction of EMT, Stat5 promoted a cancer stem cell (CSC) like PC phenotype with increased sphere formation concurrent with induction of markers associated with CSC-like properties such as CD44, Sox2, and BMI112. We further showed that activated Jak2-Stat5 axis induced metastases formation of PC in vivo in mice, which was accompanied with increased Twist1 and BMI1 in the metastases sites aligning with the in vitro results. Collectively, our findings showed that activated Stat5 signaling causes functional changes of metastatic PC cell phenotype with stem-like cell characteristics.

While ENZ is known to inhibit proliferation of PC cells through its inhibitory action on AR, ENZ was recently demonstrated to paradoxically promote development of a metastatic PC cell phenotype with increased metastatic behavior such as cell migration, invasion, anchorage-independent growth and heterotypic cell adhesion. ENZ-induction of development of dedifferentiated metastatic PC cell phenotype is likely a critical component of development of ENZ-resistance of PC.

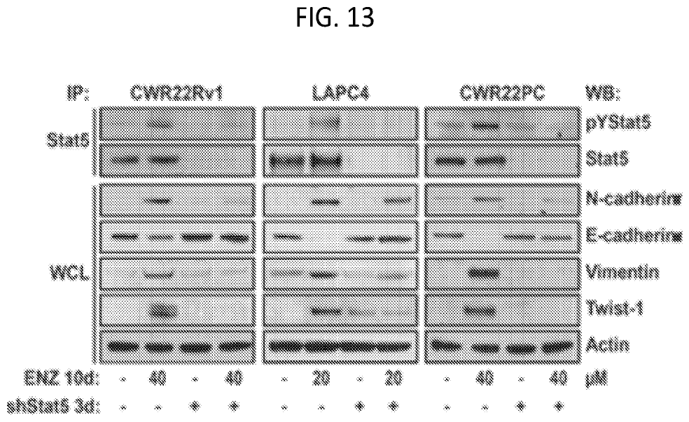

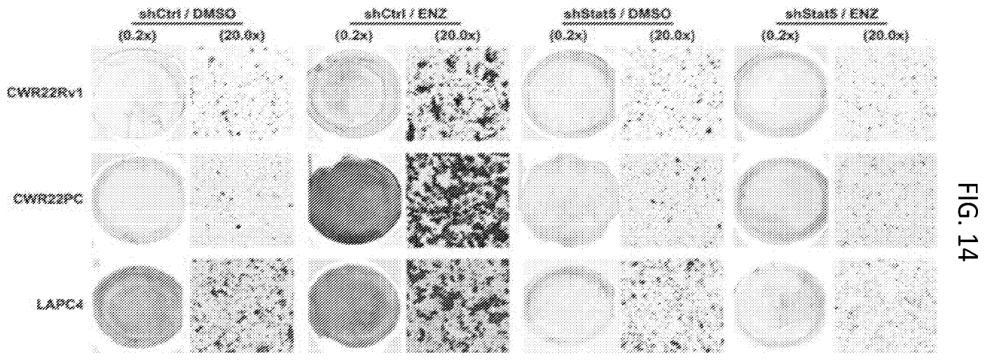

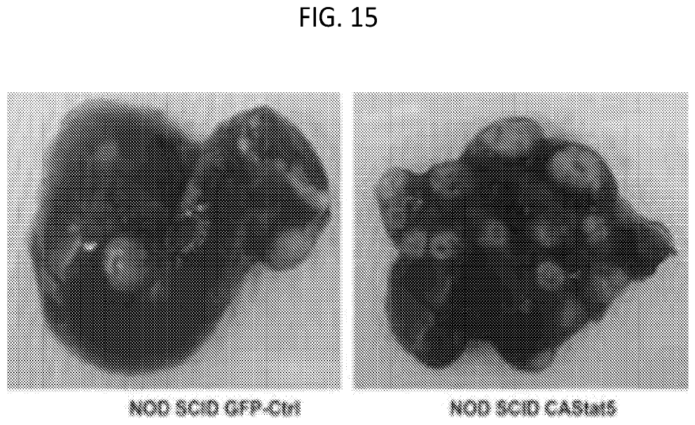

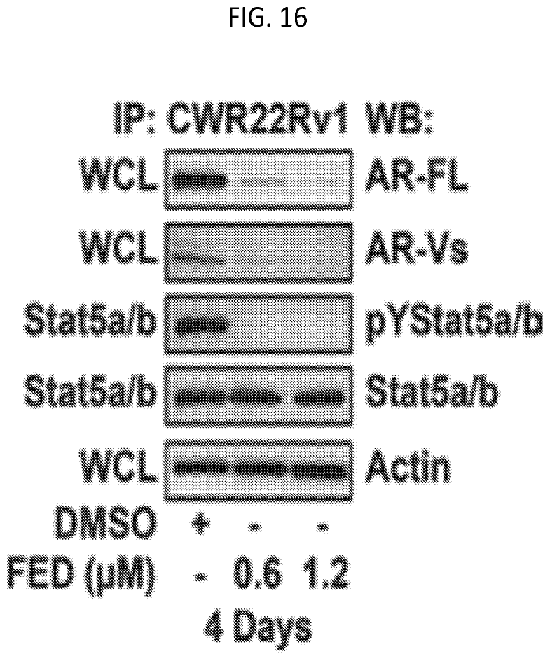

To evaluate if ENZ induces EMT and migration of PC cells in our hands, three different PC cell lines were treated with ENZ for 12 days and analyzed for the expression of EMT markers (N-cadherin, Vimentin and Twist1) by immunoprecipitation (IP) and western blotting (FIG. 13). ENZ induced the expression of N-cadherin, Vimentin and Twist1, while suppressing E-cadherin expression in all cell lines (FIG. 13). At the same time, genetic suppression of Stat5 by lentiviral expression of shStat5 blocked the ENZ induction of the EMT-markers, which suggests that Stat5 is critical for ENZ-induced EMT in PC. Importantly, ENZ induces a robust migration of three PC cell lines as detected by Boyden chamber assays (FIG. 14). The ENZ-induced PC cell migration was inhibited by genetic ablation of Stat5 by lentiviral expression of shStat5 vs. shCtrl (FIG. 14). Human CWR22Pc cells, when inoculated to the prostates of immunocompromised mice, form distant metastases in the liver in 8 weeks after orthotopic growth. As demonstrated in FIG. 15, lentiviral expression of constitutively active Stat5 (CAStat5) in PC cells dramatically increases the formation of liver metastases of PC and development of a systemic disease. In conclusion, ENZ induces both markers of EMT and a migratory phenotype which is a functional endpoint of a mesenchymal metastatic cell phenotype and can be blocked by suppression of Stat5.

### Example 5—Pacritinib and Fedratinib Suppress AR Expression in Models of Prostate Cancer

The inventor has shown that Jak2-Stat5 signaling is critical for sustaining AR mRNA and protein expression in prostate cancer (PC). Specifically, they demonstrated that pharmacological inhibition of Jak2 by pacritinib suppresses AR expression at the mRNA and protein level in PC and induces a robust decrease in PC cell viability (see Example 2). In the present Example, they test the ability of pharmacological inhibition of Jak2 by the Jak2 inhibitors fedratinib (Celgene BMS) and pacritinib to suppress AR expression in several models of PC, and demonstrate that the effect of these inhibitors is mediated by Stat5.

Importantly, safety testing of these drugs is already well underway: pacritinib is in late clinical development for myelofibrosis while fedratinib is already FDA-approved for the treatment of myelofibrosis. Further, a clinical trial testing the efficacy of pacritinib from CTI BioPharma for growth inhibition of non-metastatic castrate-resistant prostate cancer will be starting (pending IRB approval) at the MCW Cancer Center. If pacritinib shows clinical efficacy in reducing the rising PSA in advanced prostate cancer patients in this clinical trial, there will be strong interest in a follow-up or simultaneous trial looking at the efficacy of fedratinib in prostate cancer therapy.

### Example 5A: Efficacy of Jak2 Inhibition by Clinical Grade Pacritinib (PAC) in Suppressing Stat5 Phosphorylation and Androgen Receptor (AR) Expression in Prostate Cancer Cell Lines

Study 5A.1.

This study confirms that inhibition of Jak2 kinase by clinical grade PAC decreases phosphorylation of Stat5 and expression of AR in androgen dependent PC cell lines (CWR22Pc, LAPC4) as efficiently as pacritinib obtained from MedChem Express (i.e., research grade pacritinib).

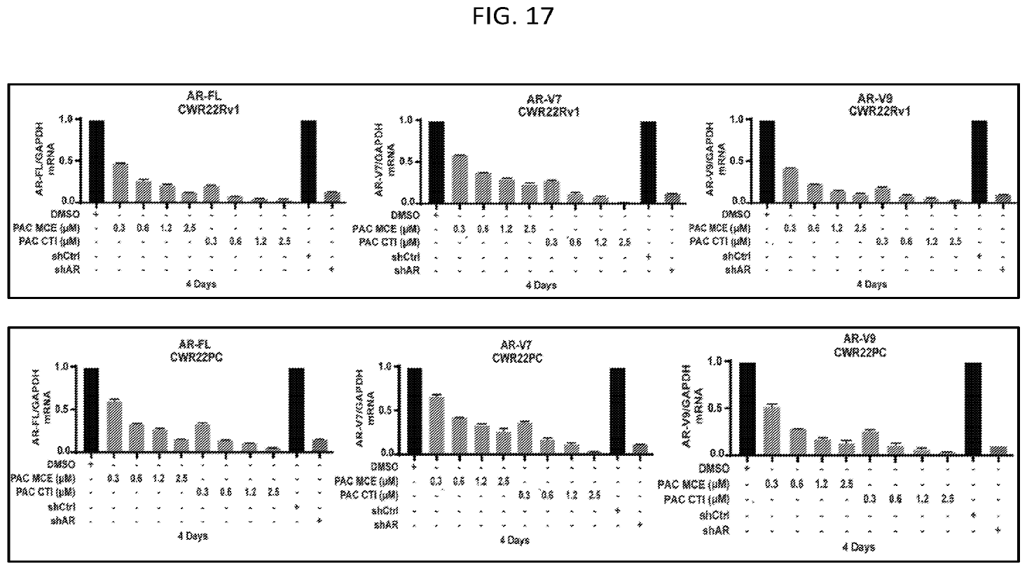

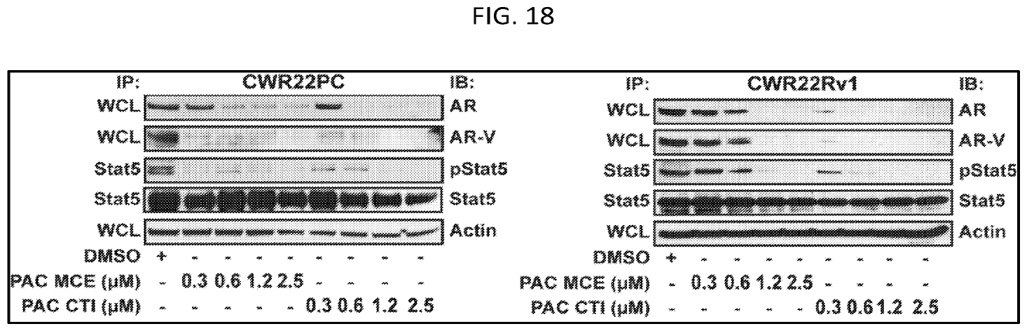

The ability of clinical grade pacritinib (CTI Biopharma) to inhibit Stat5 phosphorylation and suppress AR expression in prostate cancer cells in vitro was compared to that of research grade pacritinib (MedChem Express (MCE)) and to cells in which Jak2 was knocked down using lentivirus expressing Jak2-shRNA (shJak2). FIG. 17 shows that the results generated using research grade pacritinib were reproducible using clinical grade pacritinib, in that clinical grade pacritinib reduced mRNA expression levels of both full-length (AR-FL) and variant (AR-V7 and AR-V9) forms of AR in the PC cell lines CWR22Rv1 (top panel) and CWR22PC (bottom panel). FIG. 18 shows that clinical grade pacritinib also caused a reduction in AR expression at the protein level in these cell lines, similar to the research grade pacritinib from MedChem Express. Thus, clinical grade pacritinib induces the same effects as research grade pacrtinib, suppressing AR levels and reducing PC cell viability.

Study 5A.2:

Jak2 inhibitors pacritinib (PAC) and fedratinib (FED) suppress AR expression in androgen-sensitive and CR preclinical models of prostate cancer.

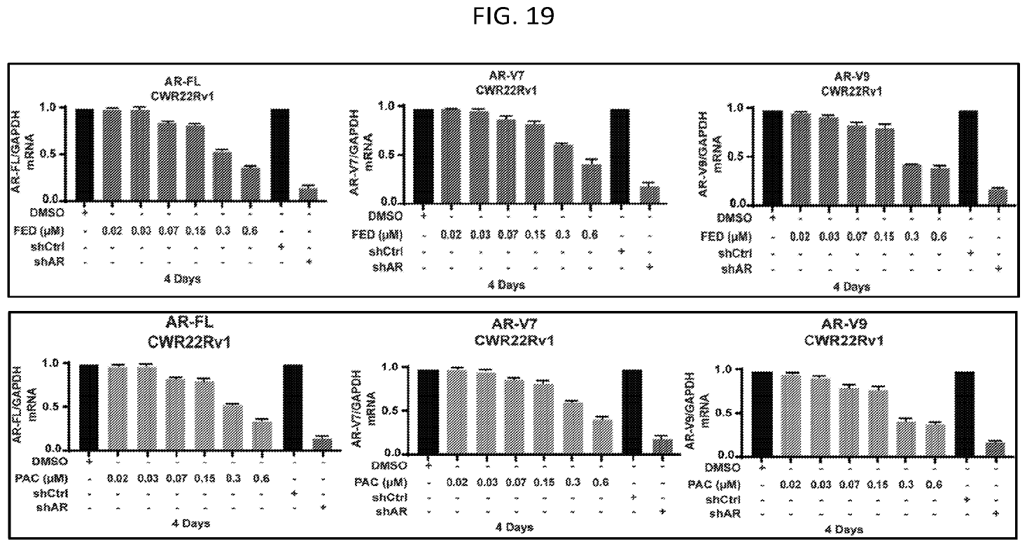

As a preliminary study, we tested whether low contrations of FED and PAC (0, 0.01875, 0.0375, 0.075, 0.15, 0.3, 0.6 μM) reduce mRNA expression of full-length (AR-FL) and variant (AR-V7 and AR-V9) forms of AR. We found that AR expression levels decrease with increased concentrations of FED and PAC (FIG. 19).

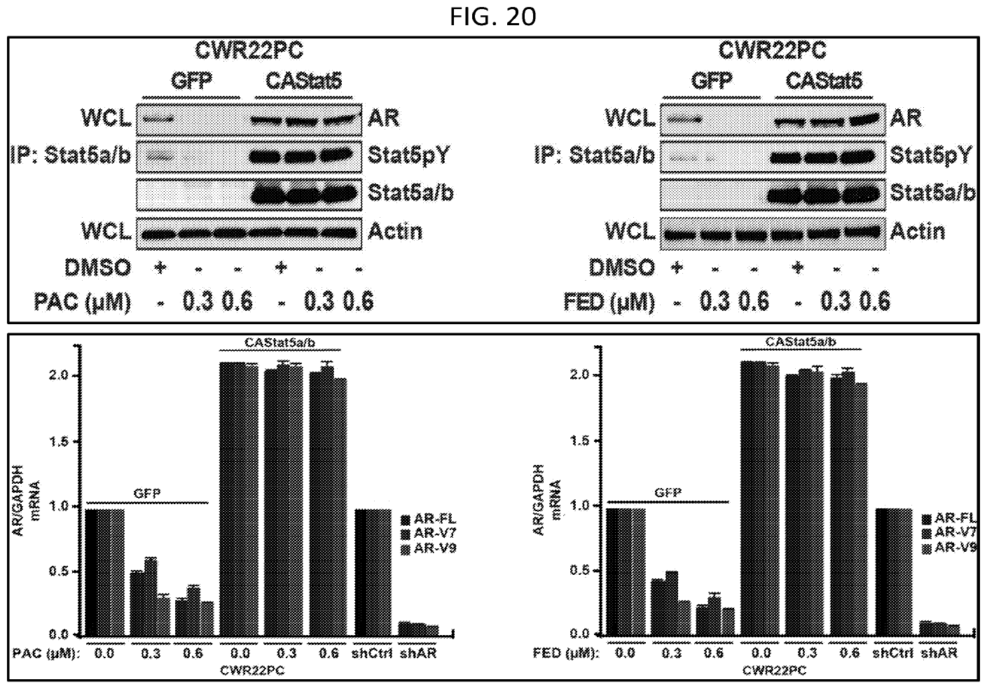

Further, we demonstrated that lentiviral overexpression of constitutively active (CA) Stat5 in CWR22PC PC cells counteracts the inhibition of AR by PAC and FED at both the protein and mRNA level (FIG. 20), which indicates that Stat5 mediates the effect of the Jak2 inhibitors PAC and FED.

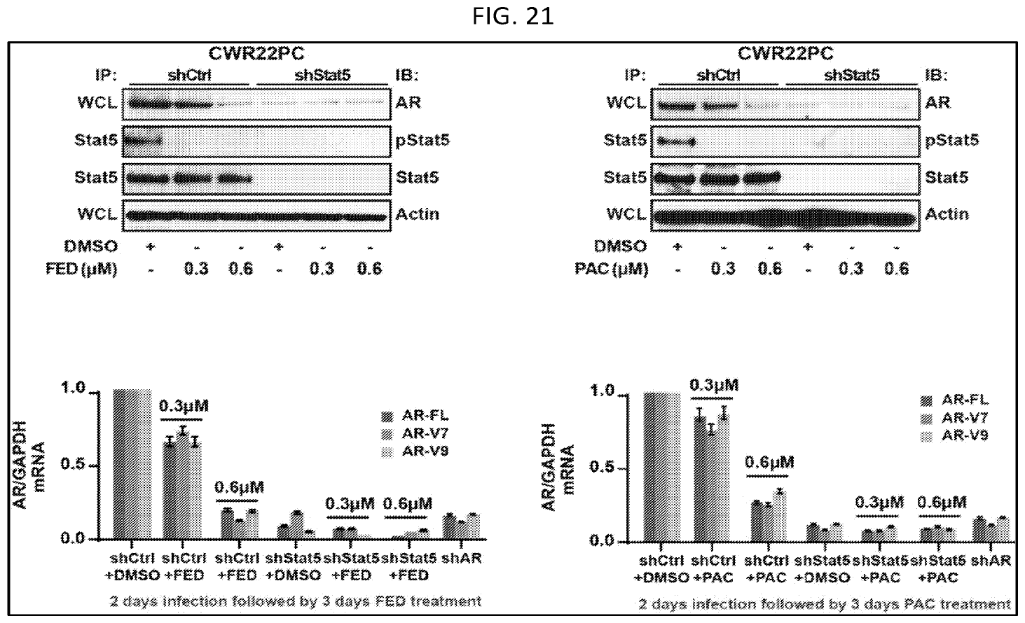

We tested whether PAC and FED are able to further reduce AR mRNA and/or protein expression following genetic suppression of AR gene expression via lentiviral expression of Stat5-shRNA in CWR22PC cells. We detected no further decrease in AR expression levels (FIG. 21). These results indicate that the effect of these Jak2 inhibitors on AR levels is mediated by Stat5 in prostate cancer cells.

Study 5A.3. PAC and FED Reduce AR Expression in Patient-Derived PCs Cultured Ex Vivo in Tumor Explant Cultures.

In a future study, we will confirm the in vitro results demonstrated above in patient-derived prostate cancer tissue explants ex vivo by assessing whether treatment with FED reduces expression of AR at the mRNA level using PCR. Additionally, we will perform an immunohistochemical analysis to determine how treatement with FED affects the levels of active Stat5 and AR protein.

### Example 5B: Effect of Jak2-Stat5 Inhibition by PAC and FED to that of Androgen-Deprivation Therapy (ADT) on PC Cell Viability and Growth in Androgen-Sensitive and CRPC Cells, Tumors, and Patient-Derived Clinical PCs Ex Vivo

Study 5B.1.

Efficacy of PAC and FED in blocking the growth of ENZ-resistant PC cells and in reducing AR mRNA/protein expression.

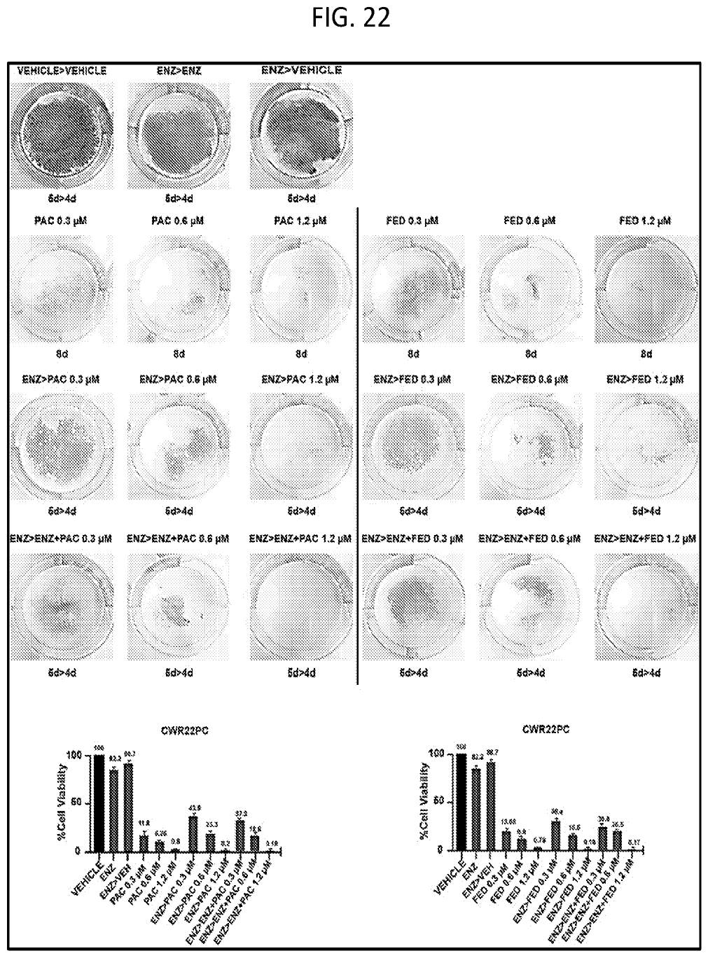

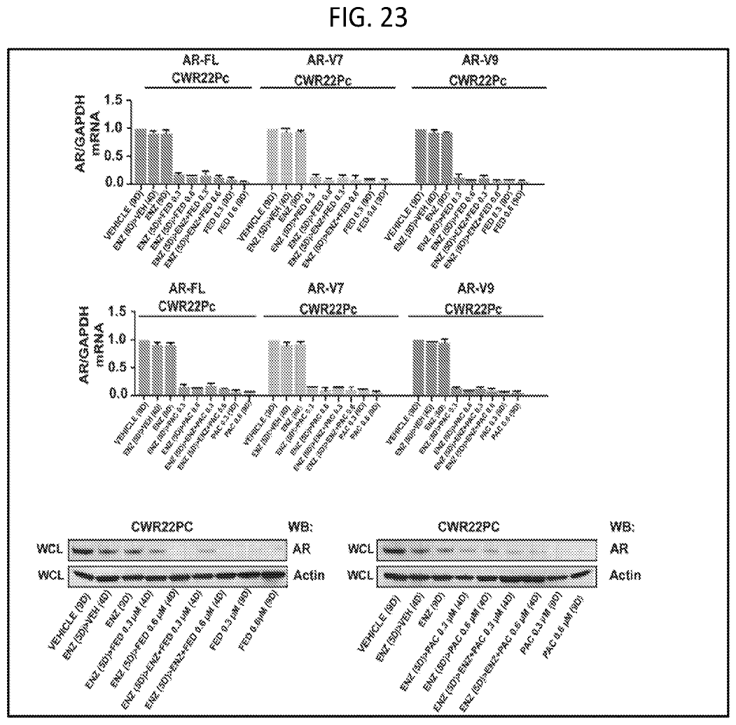

The effect of PAC and FED in blocking the growth of PC cells that survive treatment with the anti-androgen enzalutamide (ENZ) was tested in CWR22PC PC cells. The PC cells were treated in culture with ENZ (or vehicle) for 5 days, and were then treated with ENZ (40 μM), vehicle, PAC (300, 600 or 1200 nM), or FED (300, 600 or 1200 nM) for 4 days. When we compared the viability of PC cells treated with FED/PAC after ENZ treatment to that of PC cells treated with either ENZ (ENZ>ENZ) or Vehicle (ENZ>VEHICLE), both Jak2 inhibitors were able to block the growth PC cells surviving ENZ treatment with very high efficacy at a low concentration (300 nM; FIG. 22). Further, we demonstrated that this FED/PAC treatment reduces expression of AR at both the mRNA and protein level in PC cells that survive ENZ treatment (FIG. 23). Thus, PAC and FED can be used to reduce ENZ resistant PC cell growth.

Study 5B.2.

Compare the effects of PAC/FED and androgen deprivation therapy (ADT) on PC cell viability and growth in vitro.

a) Compare the Effect of PAC/Vehicle/shJak2/shCtrl to ADT Carried Out by Androgen Withdrawal on Androgen-Responsive PC Cell Viability During a Time-Course of 2-10 Days.

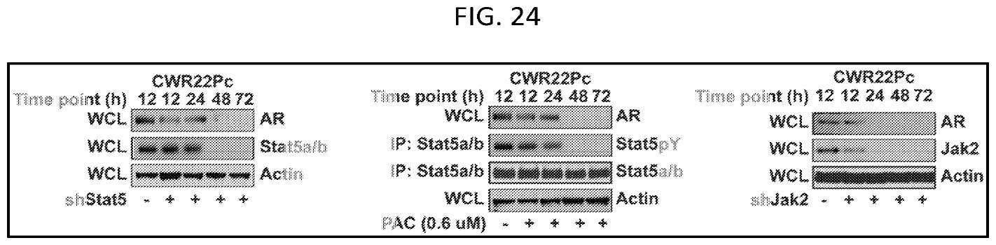

First, to determine when how long it would take to see the effects of PAC treatment on AR expression levels, we assayed AR protein levels in CWR22PC cells after treatment with PAC, vehicle, or lentivirus expressing Jak2-shRNA (shJak2) or control shRNA (ShCtrl) at timepoints spanning 1.5 days (12, 24, 36, 48, 60, 72 h) and found that AR protein levels start to decrease 48 hours after PAC treatment or 12-24 hours after genetic knockdown of Stat5/Jak2 (FIG. 24).

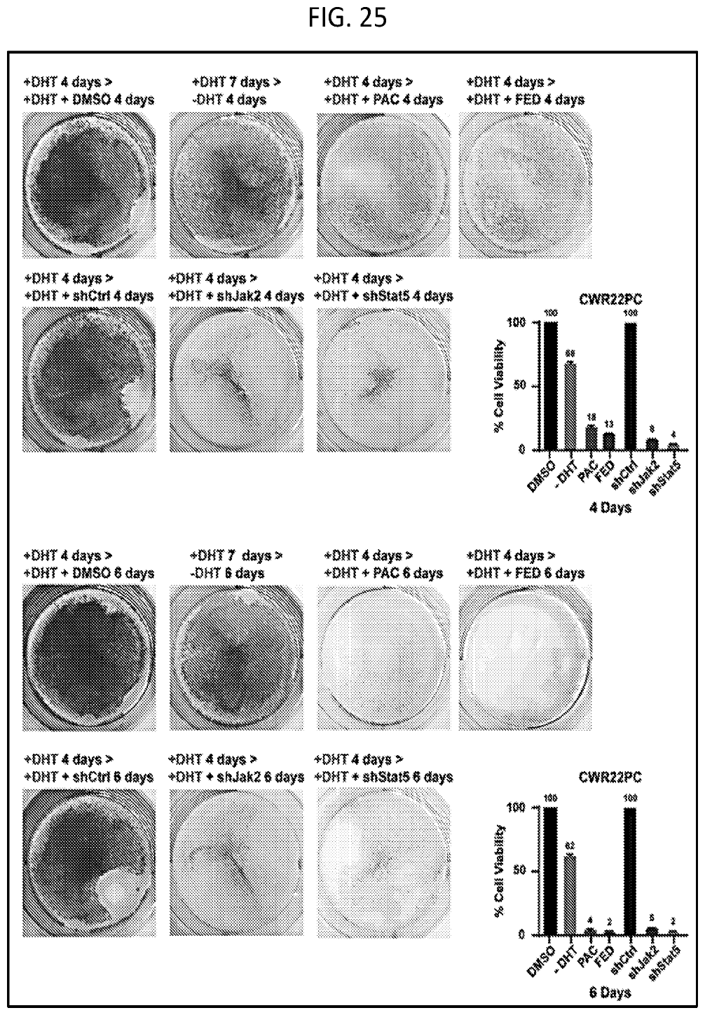

Next, we compared the effect of androgen deprivation therapy (ADT) carried out by androgen withdrawal, which is the standard therapy used to treat advanced prostate, to the effect of PAC and FED on androgen-responsive PC cell viability over a time-course of 2-10 days using crystal violet staining and cell counting. Based on this analysis, ADT decreased cell viability by 30% compared to control (+DHT), PAC decreased cell viability by 82% after 4 days of treatment and by 96% after 6 days of treatment compared to the control (+DMSO), FED decreased cell viability by 87% after 4 days of treatment and by 98% after 6 days of treatment compared to control (+DMSO), Jak2 knockdown (shJak2) decreased cell viability by by 92% after 4 days of lentivirus infection and by 95% after 6 days of lentivirus infection compared to control (shCtrl), and Stat5 knockdown (shStat5) decreased cell viability by 96% after 4 days of lentivirus infection and by 98% after 6 days of lentivirus infection compared to control (shCtrl) (FIG. 25). Thus, blocking AR with FED or PAC was shown to decrease the cell viability with higher efficacy than androgen withdrawal (ADT).

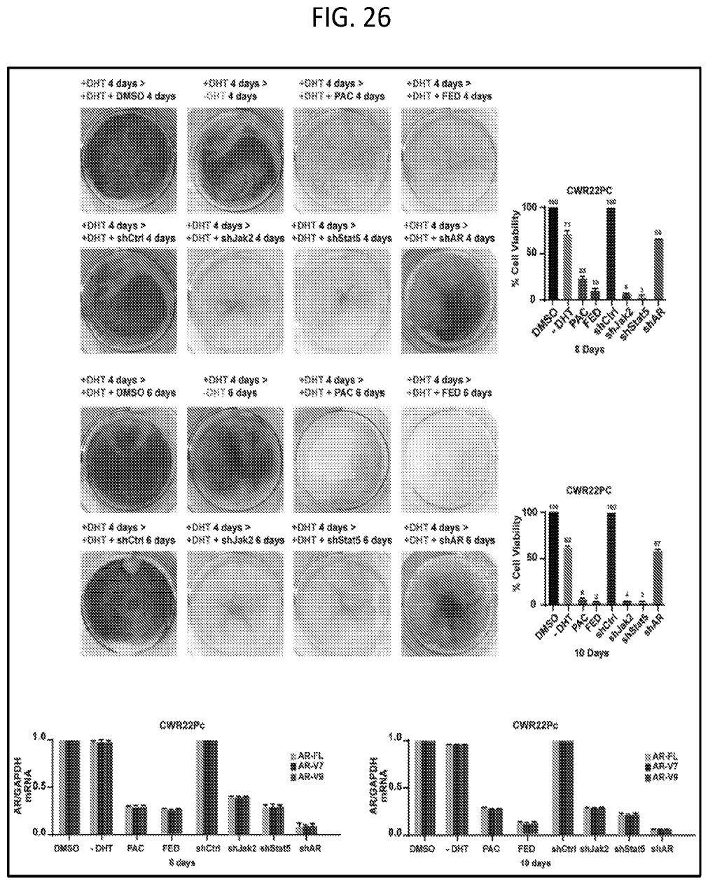

These results were confirmed when this experiment was repeated using knockdown of AR (ShAR) as a control (FIG. 26). Here, shAR decreased PC cell viability by 34% after 4 days of lentivirus infection and by 43% after 6 days of lentivirus infection compared to control (shCtrl).

Thus, we demonstrated the FED or PAC can reduced cell viability with higher efficacy than ADT treatment, and thus, can be used for treatement of patients resistant to ADT therapy.

b) PAC/FED Effect in CWR22PC Cells is Stat5 Dependent.

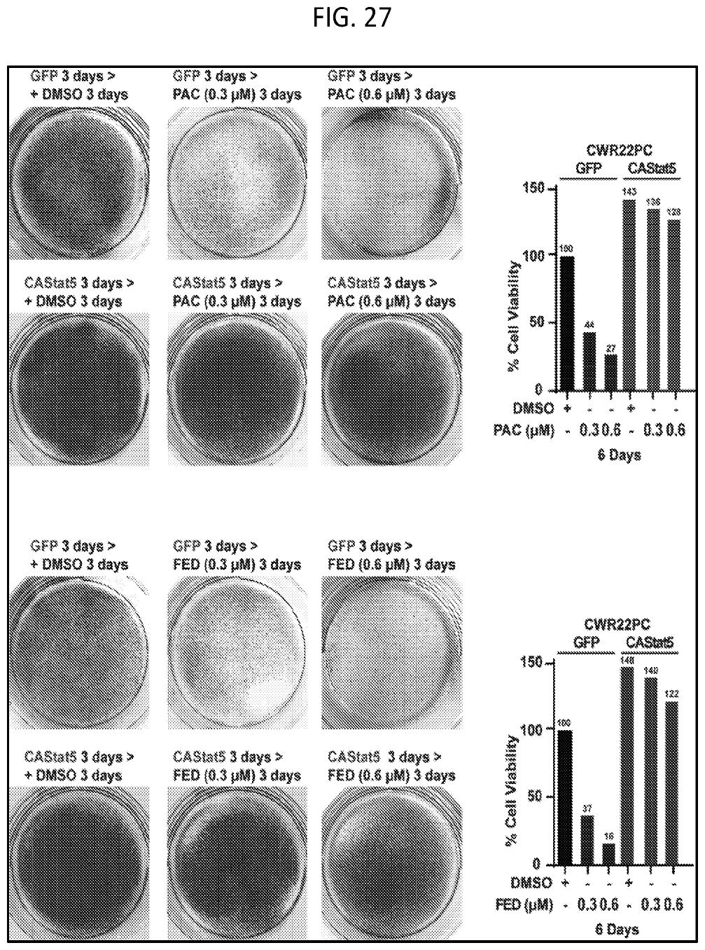

The effect of overexpressing constitutively active (CA) Stat5 (i.e., using lentivirus) on cell viability was assessed using crystal violet staining and cell counting. Here, it was found that CA Stat5 abolishes the PAC/FED-induced reduction in prostate cancer cell viability (FIG. 27). This indicates that the effect of these Jak2 inhibitors is mediated by their reduction of Stat5 expression.

Study 5B.2. Effect of PAC/FED to ADT on the Growth of Patient-Derived PCs Ex Vivo in Tumor Explant Cultures.

This experiment will demonstrate that the results obtained from PC cell culture lines is also true in patient-derived PCs. Patient-derived PCs (n=10) will be cultured ex vivo in tumor explant cultures and treated with vehicle, PAC, FED or ENZ. We will compare the efficacy of PAC or FED to ENZ or androgen withdrawal in inducing cell death (i.e., using a Caspase-3 detection assay). Additionally, part of the explants will be analyzed for AR mRNA and protein expression. We expect that PAC to reduce AR expression and have increase efficacy in PC cell killing as compared to ENZ.

Study 5C.1. Compare the Effect of PAC/FED and ADT on the Growth of PC Xenograft Tumors In Vivo.

Androgen-responsive PC cell lines (CWR22Pc, LAPC4) will be grown as subcutaneous xenograft tumors in non-castrated nude mice. Once the tumors have been established, the mice will be treated with PAC, FED, ENZ, or androgen withdrawal. Study endpoints will be serum PSA, tumor regression, cell viability and Stat5 activation status in the tumors. Additionally, parts of the tumors will be analyzed for AR mRNA and protein expression as described in the examples. We expect that both PAC and FED will suppress PC xenograft tumor growth more effectively than ADT or ENZ.

Study 5C.2. Evaluate the Effect of PAC/FED on the Expression of AR in Androgen-Dependent Prostate Cancer Xenograft Tumors in Nude Mice.

We will determine if PAC/FED will suppress AR mRNA and protein expression in PC xenograft tumors grown in nude mice in the presence of androgens. We expect that both PAC and FED will suppress AR mRNA and protein expression in PC xenograft tumors.

### Example 6—Fedratinib as a Salvage Therapy for Enzalutamide/Apalutamide/Darolutamide-Resistant Prostate Cancer

The following Example aims to address the clinical problem that after failure of second-generation anti-androgens, AR is still expressed and remains active in the majority of CRPC, driving resistance and continued tumor growth and metastases formation, ultimately causing patient death. This first-in-the field studies, we are developing a therapeutic strategy to block CRPC growth and metastatic dissemination by eliminating the expression of FL-AR and AR-Vs to nearly non-existent levels by blocking Jak2 signaling in ENZ-resistant (ENZ-R) CRPC using the Jak2 inhibitor fedratinib (FED).

Specifically, we will evaluate the efficacy of FED in suppressing growth of established ENZ-resistant PC. We will further evaluate the efficacy of FED in blocking ENZ-induced metastatic dissemination of PC. We will investigate the in vivo toxicity of ENZ in combination with FED in mice, and whether Stat5 activation status of ENZ treated patient-derived PCs in tumor explant cultures predicts responsiveness to Jak2-inhibition by FED. It is important to note that no previous clinical trials utilizing Jak2 inhibitors have been conducted in the PC space for ENZ-resistant PC. In addition, the identification of Stat5 as a key regulator of the AR gene transcription including the AR-Vs is entirely new. The proposed project is original and innovative and is expected to translate to a proof-of-concept phase I/II clinical study for Jak2 inhibitors in ENZ-resistant CRPC.

The proposed work is highly innovative because of the following conceptual innovations: (1) The concept of Jak2 as a key driver of full-length (FL) AR and AR splice variant (AR-V) expression at mRNA and protein levels in PC and growth of PC is novel and first-in-the-field. (2) The concept of ENZ and APA as drivers of Jak2-phosphorylation and increased Jak2-Stat5 signaling is new. (3) The concept of using the Jak2 inhibitor fedratinib as a strategy to block FL-AR and AR-V expression and ENZ-resistant CRPC growth is a new therapeutic approach for lethal Enzalutamide (ENZ)/Apalutamide (APA)-resistant PC. (4) The concept of pharmacological inhibition of Jak2 signaling by fedratinib as a therapeutic strategy to suppress both growth and metastatic dissemination of PC during ENZ/APA treatment is new and has high translational significance for therapy development for PC patients. (5) The concept of pharmacological inhibition of Jak2 signaling by fedratinib as a therapeutic strategy to broadly suppress the expression of FL-AR and diverse AR-V species and proliferative and metastatic PC cell phenotype has high translational significance for therapy development for PC patients. (6) Jak2 control of AR expression in PC provides a conceptual advance that Jak2 inhibitors could represent “3rd generation anti-androgens” with broader and more durable effects on AR signaling than the 2nd generation agents ENZ/APA. (7) We will develop a strategy to improve therapeutic response of PC to ENZ by blockade of Jak2-Stat5 signaling by fedratinib and/or provide a second-line therapy for ENZ-resistant PC. The proposed work will establish the Proof-Of-Concept (POC) in preclinical PC models for efficacy of FED to block ENZ-resistant CRPC growth and is expected to be followed by a POC first-in-the-field phase I/II clinical trial to test the efficacy of FED in suppressing growth of Enzalutamide-resistant CRPC in patients.

We conducted a pilot experiment to test the efficacy of FED in suppressing AR levels in PC cells (FIG. 16). Our data show that FED depletes FL-AR and AR-V protein levels almost entirely in CWR22Rv1 cells (FIG. 16). In conclusion, pharmacological Jak2 inhibitors potently suppress AR levels and viability of CRPC cells.

Experimental tools: A) For in vitro PC studies, we will use CWR22Pc, LAPC4, VCaP, and LNCaP PC cells. B) To test the efficacy of Jak2-Stat5 inhibitors in blocking Enzalutamide-resistant growth in vivo, androgen-dependent human CWR22Pc model of CRPC progression in athymic nude mice as subcutaneous tumors(51) will be used, and offers 100% take rate and consistent formation of CR tumors within 30-40 days of androgen withdrawal. Also, LAPC4 cells, which are tumorigenic in nude mice will also be used. C) For patient-derived clinical PCs, ex vivo 3D tumor explant culture system will be used, developed by the PI(18, 24, 39, 52-54), to test the proposed hypotheses in patient-derived PCs.

### Example 6A: Pre-Clinical Efficacy of FED in: a) Preventing Development of ENZ-Resistance of PC and b) Suppressing Growth of Established ENZ-Resistant PC

Rationale: Jak2 signaling drives AR-FL/V7/V9 mRNA expression in PC, and sustains viability and growth of PC cells in vitro and in vivo. At the same time, ENZ-induces Jak2-Stat5 signaling and AR expression in PC.

Approach: We will evaluate the efficacy of pharmacological Jak2 inhibition by FED a) to prevent development of ENZ resistance when given as a front-line combination therapy with ENZ, and b) to suppress the AR and growth of CRPC after ENZ-resistance has developed. These preclinical studies will utilize PC cell lines in vitro, human xenograft tumors in vivo and in 3D tumor explant cultures of clinical patient-derived PCs ex vivo to model ENZ-resistant PC growth.

6A.1. Evaluate the Efficacy of FED in Suppressing AR mRNA/Protein Expression and ENZ-Resistant CRPC Growth a) In Vitro and b) Ex Vivo in Patient-Derived PC in Tumor Explant Cultures.

A) Evaluate the Efficacy of FED to i) Prevent Development of ENZ-Resistant PC Growth or ii) as a Second Line Treatment after ENZ in PC Cell Lines Cultured In Vitro:

i) Androgen-sensitive PC cell lines will be cultured for 20 days with vehicle alone, ENZ alone or in combination with FED vs. vehicle, followed by analyses described below.

ii) In the second set of experiments, androgen sensitive and ENZ resistant PC cell lines will be cultured with ENZ vs. vehicle for 20 days. In the second phase after prolonged ENZ-treatment, Jak2 will be pharmacologically inhibited by FED alone or in combination with ENZ, using increasing concentrations of FED and a time-course ranging from 5-10 days. The control groups for both sets of experiments include vehicle, lenti-shJak2 and lenti-shCtrl. The control cell line PC-3 (AR−/Stat5−) is not expected to respond to ENZ or FED.

At the end of the experiment, the AR mRNA and protein levels will be analyzed by q-RT-PCR and D3. The fraction of apoptotic cells will be determined by DNA fragmentation ELISA assay and Caspase-3 assay³⁴. Cell growth and cell cycle distribution will be evaluated by FACS, MTT, crystal violet staining and clonogenic survival assays, as we have demonstrated previously(52). We expect that Jak2 inhibition by FED will prevent development of ENZ-resistant P C cell growth. Also, we expect that FED will effectively reduce the AR levels and the fraction of viable PC cells surviving ENZ treatment.

B) Evaluate f FED+/−ENZ Reduces AR Levels and Growth of Patient-Derived 3D Explant Cultures:

Fresh surgical PC tissues from 50 patients will be cultured ex vivo in 3D tumor explant cultures in the presence of androgens, as we have described^{13,14,38,63,81-86.} After the tissues adapt to the culture condition by day 4, we will treat the explants with ENZ or vehicle control for 20 days. One third of the tissue explants will be harvested for analysis of Stat5 activation status. On day 24, the rest of the PC explants will be exposed to FED, ENZ+FED vs. vehicle in each group for 7 days. After the treatment, we will evaluate if FED decreases the levels of AR mRNA and protein using quantitative RT-PCR, immunoblotting and IHC. Cell death will be determined by TUNEL staining, proliferative indexes will be evaluated by IHC for PCNA/Ki67. For IHC and TUNEL analyses, we will use an advanced automated IHC analysis technology which utilizes multiplex immunofluorescence histocytometry by Tissue Studio image analysis software, previously described and validated. We expect that FED will suppress AR mRNA and protein expression and induce apoptotic death of ENZ-treated patient-derived PC tissue explants.

Statistics (a and b): Analysis of variance followed by Tukey-Kramer test will be used to test for differences among treatments with respect to logit-transformed fraction of positive cells.

Experiment 6A.2. Evaluate the Efficacy of FED in Eliminating AR-FL/V7/V9 mRNA and Protein Expression in PC and Suppressing Development of ENZ-Resistance and/or Suppressing Growth of ENZ-Resistant CRPC.

A) Determine the Efficacy of FED in Suppressing ENZ-Resistant CRPC Xenograft Tumor Growth as a Second-Line Treatment Alone or in Combination with ENZ (Phase II):

PC cells (CWR22Pc, LAPC-4) will be injected s.c. in nude mice supplemented with DHT pellets, and the mice will be treated daily for 20-25 days with ENZ (30 mg/kg; p.o.) vs. vehicle. In a parallel set of experiments, CWR22Pc-ENZ-R and LAPC4-ENZ-R cells will be inoculated s.c. in nude mice treated with ENZ. Once ENZ-resistant tumor growth emerges (10 mm diameter), the mice will be treated for an additional 20-25 days (Phase II) with vehicle, FED (50, 100, 150 mg/kg/d p.o.) alone or in combination with ENZ, with continued ENZ as control group for 15-30 days. Each treatment group includes 15 mice based on the power calculations (see below). This includes 5 extra mice in each group for prolonged treatment with FED or FED+ENZ to evaluate if late recurrence would occur. Also, we will include additional 5 mice to the groups of vehicle and FED (100 mg/kg) for the sc-RNA-Seq. The tumors will be evaluated for AR and Stat5 protein levels by IHC and IB, and for FL-AR and AR-V mRNA levels by quantitative RT-PCR. Tumors for the sc-RNA-Seq. will be flash-frozen. The tumors will be analyzed for apoptosis and proliferative indexes by TUNEL and PCNA/Ki67 IHC (Tissue Studio Analysis), and changes in tumor volumes will be measured as described(39, 52). We expect that FED will suppress AR levels and significantly suppress the growth of ENZ-resistant PC tumors compared to the other treatment groups.

Statistics: Mixed effects linear regression will be used to model the trajectory of tumor growth over time. Tumor volumes will be log-transformed in order to satisfy the assumption of normality. Separate slopes and intercepts will be assumed for each group, and a random intercept term will be included to account for correlation among repeated measurements from the same mouse.

Sample size computations: From a preliminary study, the mean tumor volume is ˜1 cm³ and the standard deviation for the control group is estimated to be 0.2 cm³ at the time of interest. Assuming that the groups have the same variation as the control group, 10 mice per group will obtain 82% power to detect 65% reduction in tumor volume (to 0.35 cm³) from the control group (1 cm³) at the 0.1% significance level (selected due to multiple comparisons in several of the experiments). This is based on tumor volumes in the log₁₀ scale.

B) Evaluate the Efficacy of Pre-Emptive Combination Therapy of FED with ENZ in Preventing Development of ENZ-Resistant CRPC Xenograft Tumor Growth when Administered in Combination with ENZ (Phase I):

PC cells (CWR22Pc, LAPC-4) will be injected s.c. in nude mice supplemented with DHT pellets. Once the androgen-driven tumors form (6-7 mm in diameter), the mice will be treated daily for 30-50 days with vehicle or ENZ (30 mg/kg; p.o.) alone or in combination with FED (50, 100, 150 mg/kg p.o.) vs. vehicle. Each treatment group includes 10 mice based on the power calculations (see above). At the time of sacrifice, the tumors will be evaluated for AR and Stat5 protein levels by IHC and IB, and for FL-AR and AR-V mRNA levels by quantitative RT-PCR. The tumors will be analyzed for apoptosis and proliferative indexes by TUNEL and PCNA/Ki67 IHC, respectively, and changes in tumor volumes will be measured as previously described. We expect that FED will significantly suppress AR levels and development of ENZ-resistant PC tumors compared to the other treatment groups.

### Example 6B: Determine the Functional Significance of Jak2-Stat5 Signaling in Mediating ENZ Induction of Metastatic Behavior of PC Cells, and Evaluate the Therapeutic Efficacy of Targeting Jak2 in Combination with ENZ to Block Metastatic Dissemination of PC in Preclinical Models of PC

Rationale: While ENZ slows PC cell proliferation, ENZ was recently shown to simultaneously induce an emergence metastatic PC cell phenotype with increased metastatic behavior. Our data show that ENZ induces robust Jak2-Stat5 signaling in biopsies of distant metastases from ENZ-treated patients, in PC cells in culture and in preclinical PC models in vivo. At the same time, Stat5 has been shown to induce EMT and metastatic behavior of PC cells in vitro and in vivo. Approach: We hypothesize that Jak2-Stat5 signaling mediates ENZ-induction of EMT and acquisition of motile and invasive properties in PC cells. We further propose that targeting Jak2 by FED will improve the therapeutic efficacy of ENZ in PC by blocking ENZ-induced metastatic behavior of PC.

Study 6B.A.1. Determine the Significance of Jak2 and Stat5 in Mediating ENZ Induction of Mesenchymal PC Cell Phenotype.

The studies proposed in this section will address the question whether genetic or pharmacological knockdown of Jak2 or Stat5 will block ENZ-induction of EMT in PC cells a) in vitro and b) in patient-derived clinical PCs ex vivo in tumor explant cultures. First, Jak2 or Stat5 will be genetically suppressed by lentiviral expression of Jak2-shRNA or Stat5-shRNA (vs. shCtrl) prior to ENZ (vs. vehicle) treatment of PC cells (Table 1) for 10 and 20 days. Control groups will be infected with lenti-virus expressing constitutively active (CA) Jak2(V617F) or CAStat5(S710F/S715F), which induce EMT of PC cells(24). The cells will be analyzed for Jak2 levels, Jak2 phosphorylation, Stat5 phosphorylation and Stat5 levels by IP and D3. The mRNA and protein levels of the EMT markers N-Cadherin, total and cell-surface E-Cadherin, Twist1, Vimentin and Fibronectin will be evaluated by q-RT-PCR and by D3. For evaluation of cell surface E-cadherin, we will biotinylate all cell surface proteins. E-cadherin will be IPed and the IPs will blotted for biotin to detect changes in the cell surface E-Cadherin levels, as previously described¹³. Second, Jak2 will be pharmacologically inhibited by FED in three different doses each vs. vehicle, and simultaneously treated with ENZ (vehicle control) for 20 days. As positive controls, Jak2(V617F) or CAStat5(S710F/S715F) will be lenti-virally expressed in control groups and compared to lenti-GFP treated cells. The EMT marker levels will be determined as described above. Third, 20 patient-derived clinical PCs will be cultured ex vivo in tumor explant cultures, as we have described previously. Tumor explants will be treated with ENZ vs. vehicle for 15 days alone or in combination with increasing concentrations of FED vs. vehicle. At the end of the culture, the explants will be analyzed for the expression of EMT markers N-Cadherin, total and cell-surface E-Cadherin, Twist1, Vimentin and Fibronectin by IB and by quantitative immunohistochemistry Specifically, we will employ advanced automated multiplex immunofluorescence histocytometry using Tissue Studio image analysis software, previously described and validated. In these three sets of experiments, we expect that both genetic and pharmacological suppression of Jak2 or Stat5 will block ENZ-induction of a mesenchymal PC cell phenotype and the expression of markers reflecting the metaplastic change. Statistics: Mixed effects model with empirical standard errors will be used to determine if levels of nuclear Stat5 and EMT markers in the treatment groups differ from controls. Mixed models will allow appropriate adjustments for repeated measurements.

Study 6B.A.2. Evaluate the Involvement of Jak2 and Stat5 in ENZ-Induction of Functional Endpoints of Metastatic PC Cell Phenotype In Vitro.

The goal of the experiments described in this section is to evaluate if ENZ induces functional endpoints of migratory and invasive PC cell phenotype to determine if Jak2 and Stat5 are critical for ENZ-induction of the cell characteristics required for functional metastasis processes including endpoints of migratory and invasive PC cell phenotype. In PC cells, Jak2 or Stat5 will be genetically suppressed by lentiviral expression of shJak2 or shStat5 in PC prior to ENZ (vs. vehicle) treatment of the cells for 10 and 20 days. CAJak2 or CAStat5 will be lentivirally expressed in the control groups (compared to lenti-GFP) and are expected to induce functional endpoints of EMT, as shown previously. Alternatively, Jak2 or Stat5 will be pharmacologically inhibited by FED simultaneously with the ENZ treatment. After treatment with ENZ for 10 and 20 days, (a) PC cell migration will be evaluated using wound filling assay and Boyden chamber assay with Fibronectin as the chemoattractant, as we have described previously^{13, 37}. Equal number of cells will be plated, and the migrated cells will be evaluated at two timepoints after plating, 6 h and 12 h, by crystal violet staining of the wells (wound filling assay) or the Boyden chamber inserts. (b) Induction of PC cell invasion by ENZ will be determined using three different matrices; fibronectin, extracellular matrix mixture (laminin, collagen I and IV) and matrigel in Boyden chambers and the number of cells capable of invading through the matrix will be evaluated by crystal violet staining and imaging. (c) ENZ induction of heterotypic adhesion of PC cells to form pre-metastatic niches will be evaluated using fibronection cell adhesion assay(24), where the cells will be plated to culture wells coated with 7 μg/ml fibronectin or poly-L-lysine, incubated for 1 h followed by crystal violet staining of fixed cells and quantification at 595 nm, as we have described. (d) ENZ induction of anchorage-independent growth will be evaluated in the classical soft-agar cell growth assay, which measures the capability of the cells to proliferate in semi-solid culture media and quantification by fluorometric detection of the colonies (Cell BioLabs). We expect that both genetic and pharmacological suppression of Jak2 or Stat5, both, will significantly suppress ENZ-induction of the functional characteristics of a metastatic PC cell phenotype.

Statistics: For cell migration, invasion and adhesion experiments, linear models will be used, while cell scattering assays will analyzed by nonparametric Wilcoxon sum tests. Mixed effects terms will be used to adjust for repeated observations while Tuckey's HSD method will be used to correct for multiplicity wherever appropriate.

Study 6B.A.3. Evaluate the Efficacy of Pharmacological Jak2 Inhibition in Blocking ENZ Induction of Distant PC Metastases and Systemic Disease.

In the studies proposed in this section, we will determine the efficacy of the Jak2 inhibitors, PAC/FED, in suppressing the formation of distant metastases of orthotopic human CWR22Pc PC tumors during ENZ treatment in mice. Luciferase-fluorescent double labelled PC cells (CWR22Pc) will be inoculated orthotopically in prostates of athymic nude mice, and the mice will be treated daily for 60 days with ENZ (30 mg/kg; oral gavage vs. vehicle). One group of the ENZ-treated mice will be treated simultaneously with PAC and another group with FED (0, 50, 100, 150 mg/kg p.o., daily)²⁹ compared to vehicle-treated mice. A positive control group will be fluorescent/luciferase labeled cells expressing constitutively active (CA) Stat5 (lenti-viral expression) which consistently form distant liver metastases vs. lenti-shCtrl-expressing cells. Each treatment group includes 10 mice based on the power calculations (see below). At the end of the treatment period, the mice will be analyzed for micro-metastases and macro-metastases formation by IVIS Spectrum CT. In addition, the livers will be analyzed for metastases formation by manual counting and imaging followed by evaluation by the ImageJ program, described previously(24). The lungs, brain and liver tissues will be analyzed for human-specific Alu DNA sequences using Alu-based real-time PCR method. Both the primary tumors and the liver metastases will be evaluated for the levels of Jak2 and Stat5 activation and protein and for the EMT markers N-cadherin, total and cell-surface E-cadherin, Twist1, Vimentin and Fibronectin by q-RT-PCR, IB and by multiplex immunofluorescence histocytometry using Tissue Studio image analysis software. We expect that PAC and FED, both, will suppress the levels of the EMT markers at transcriptomic and protein levels, Jak2-Stat5 signaling and formation of liver metastases of ENZ-treated mice compared to control groups.

Statistics: For evaluation of the number of liver metastases, statistical significance will be calculated using Wilcoxon rank-sum test.

Sample size computations: In the animal treatment studies of PC metastases formation, from a preliminary study, the mean number of CAStat5-induced metastases in the livers was 15 and the standard deviation for the control group is estimated to be 2 at the time of interest. Assuming that the groups have the same variation as the control group, 10 mice per group will obtain 82% power to detect 65% increase in metastases formation from the control group at the 0.1% significance level (selected due to multiple comparisons in several of the experiments).

### Example 6C: Evaluate a) the In Vivo Toxicity of the Combination of FED with ENZ in Mice, and b) Whether Stat5 Activation Status of PC Predicts Responsiveness to Jak2-Inhibition by FED

Moreover, if residual CRPC tumors exist after Jak2-inhibitor-induced AR depletion, c) we will evaluate the clonal composition and the predominant transcriptomic selection in the cells of the residual PC tumors utilizing single-cell RNA-sequencing (sc-RNA-seq).

Rationale: ENZ-induces Jak2-Stat5 signaling and AR expression in PC and, therefore, potential toxicity of combined administration of FED in conjunction with ENZ should be evaluated in vivo in mice. Furthermore, since ENZ-induces Jak2-Stat5 signaling in PC, which sustains viability of PC cells during ENZ treatment, positive status for Stat5 activity may serve as a predictive marker to identify those patients whose PC will respond to FED.

Approach: We will evaluate acute and chronic toxicity of the combination of the Jak2 inhibitor FED with ENZ according to the guidelines of OECD in 8 weeks old male mice. Utilizing PC samples obtained from patients, we will evaluate whether positive status for active Stat5 after ENZ-treatment predicts responsiveness of PCs to FED in 3D tumor explant cultures. Finally, if residual PC xenograft tumors after FED treatment in mice exist, we will evaluate the predominant transcriptomes and the clonal composition of PC xenograft tumors surviving FED-induced depletion of the AR by sc-RNA-seq.

Study 6C.1. Evaluate the In Vivo Toxicity of the Combination of the Jak2 Inhibitor FED with ENZ in Mice.

We will use 30 mg/kg/d (p.o.) of ENZ dose in combination of FED (50, 100, 150 mg/kg/d p.o.). The toxicity studies will be carried out according to the guidelines of OECD in 8 weeks old male mice.

I) Acute toxicity: FED will be given to male mice (n=10/group) in combination with ENZ vs. vehicle vs. FED or ENZ alone for 14 days to observe for signs of toxicity, followed by collection of blood for clinical chemistry and hematology parameters and the organs for histopathology.

II) Chronic toxicity: Male mice (n=10/group) will be given ENZ with FED vs. vehicle vs. FED or ENZ alone for 28-d dosing period.

A) Basic observations: The signs of toxic effects, mortality and acute respiratory (labored breathing, wheezing) and behavioral changes (e.g. hunched posture, lethargy, seizures, ataxia, righting reflex) will be observed every 0.5 h for after administration. Changes in body weight, food intake, drinking, appearance and ocular discharge/distinct icterus will be monitored daily. The plasma samples will be analyzed by Marshfield Laboratories (WI) for clinical chemistry.

B) Clinical biochemistry: Plasma samples will be analyzed for liver (AST, ALP, alkaline phosphatase, total bilirubin, bile acid) and kidney functions (BUN, albumin, globulin and albumin-globulin ratio).

C) Necropsy and histopathology: Brain, liver, heart, kidney and spleen will be processed for histopathological analyses for histology and ultra-structural changes.

D) Hematological analysis: A detailed hematological analysis (Versiti Blood Research Institute, MCW) will include counts of white and red blood cells, lymphocytes, monocytes, eosinophils and granulocytes and hemoglobin.

E) Neurological functions will be assessed using the Neurodeficit Score System, as described previously.

Study 6C.2. Evaluate Whether Positive Status for Active Stat5 after ENZ-Treatment Predicts Responsiveness to FED in Patient-Derived PCs in 3D Tumor Explant Cultures:

We will evaluate the explants from each patient (n=50) from Study 1.1.b. for Stat5 activation status after the treatment with ENZ utilizing IHC (Tissue Studio Analysis) and IB, as we have demonstrated, and the status will be categorized as positive (scores 2-3) or negative (scores 0-1). Responsiveness to FED vs. vehicle will be also categorized as responsive vs. non-responsive, where meeting the responsive status requires induction of apoptosis in at least 50% of the tissue. We expect that positive Stat5 activation status in response to ENZ in tumor explant cultures of clinical PCs will predict responsiveness to FED.

Statistics: The agreement between the Stat5 status and FED response will be measured using the Cohen's kappa statistic and general descriptive measures such as percent concordant and percent discordant. A minimum of 32 subjects are required to detect statistically significant kappa of 0.6 or higher (usually indicative of moderate to significant agreement) versus a null hypothesis kappa of 0.2 or lower (indicative of no agreement) at the 0.05 level of significance with at least 80% statistical power.

Study 6C.3. Evaluate the Predominant Transcriptomes and the Clonal Composition of PC Xenograft Tumors Surviving FED-Induced Depletion of the AR by Sc-RNA-Seq.

(a) Sc-RNA-seq of CWR22Pc cell xenograft tumors treated with vehicle or FED in mice: Residual CWR22Pc tumors will be homogenated to cell suspension followed by enrichment and isolation of PSA expressing human PC cells using flow cytometry. CWR22Pc cells will be sorted into individual cells and the cells will be encapsulated and barcoded for library preparation using the 10× Genomics microfluidics system, followed by sequencing. ScRNA-Seq data will be demultiplexed, aligned to reference human genome mm10 and UMI-collapsed using the Cell Ranger software (version 3.1.0). Cells with fewer than 200 or larger than 4000 detected genes will be eliminated to exclude cell debris or doublets. Cells with large percentage (larger than 25%) mitochondrial-related genes will also be eliminated to remove dying or dead cells.

(b) Data analyses and integration: Gene expression profiles in 10-20,000 cells will be examined by the unbiased UMAP analysis to group cells with similar transcriptional profiles into one cluster and thus to resolve the cellular heterogeneity of residual CRPC tumors surviving dual FED-induced AR depletion in ENZ-R setting. Monocle 2, a machine-learning algorithm known as reverse graph embedding (RGE), will be used to construct cell differentiation trajectories^{95,96} and thus reconstruct the evolutionary trajectories of PC cells. Lastly, based on the databases, such as the Neuroendocrine Differentiation (NE) transcriptomes, stem-like cell transcriptomes and the GOTZMANN EMT UP-gene set, the qualitative transcriptional profiles among the different cell clusters will be compared to identify rare subclones with NE or EMT features and possible stem cell-like properties. Each tumor will be analyzed and reported individually in addition to the identification of the most common transcriptomic features between the tumors in the treatment groups.

### Example 7—Use of Jak2-Stat5 Inhibitors of a Second-Line Therapy for Apalutamide-Resistant Prostate Cancer

The following Example aims to address the clinical problem that after failure of ENZ and Apalutamide, AR is still expressed and remains active in the majority of CRPC and drives resistance and continued tumor growth, ultimately causing patient death⁵. We have demonstrates that ENZ induces hyperactivated Jak2-Stat5 feed-forward in PC72. As shown in the Examples, our data indicate that Stat5 is a potent positive regulator of AR mRNA and protein expression in PC cells grown in vitro and in vivo, and in patient-derived PCs cultured ex vivo. Importantly, Stat5 also maintains the expression of AR-V7 and AR-V9 in PC, two constitutively active AR variants that are associated with clinical resistance of PC to ENZ. In reverse experiments, our data show that inhibition of Jak2-Stat5 signaling pathway block AR expression in PC significantly suppresses growth.

We propose that Apalutamide induces Jak2-Stat5 signaling in PC and, by pharmacological inhibition of Jak2-Stat5 signaling by the new-generation Jak2 inhibitors will suppress Apalutamide-resistant PC growth. We further propose that pharmacological inhibition of Jak2-Stat5 signaling will suppress AR expression in Apalutamide-resistant PC. The goal of the proposed work is to identify the Jak2-inhibitors with highest efficacy in blocking growth of Apalutamide-resistant PC in preclinical models. The success with the proposed work is expected to lead to identification of the most potent Jak2 inhibitors for blocking Apalutamide-resistant PC growth, and position us for a first-in-the-field phase I/II clinical trial to test the efficacy of the selected Jak2 inhibitors in suppressing growth of Apalutamide-resistant CRPC.

The work proposed is highly innovative because of the following conceptual innovations: (1) The concept of Enzalutamide and Apalutamide as drivers of Jak2-phosphorylation and increased Jak2-Stat5 signaling is new. (2) The concept of Jak2 as a key driver of full-length (FL) AR and AR splice variants (AR-V) expression at mRNA and protein levels in PC is novel and first-in-the-field. (3) The concept of using the Jak2 inhibitors as a strategy to block FL-AR and AR-V expression in ENZ-resistant CRPC growth is a new therapeutic approach for lethal Apalutamide-resistant PC. (4) The concept of Jak2-Stat5 signaling as a mediator of Apalutamide-induced metastatic and proliferative PC cell phenotype with increased capacity to invade neighboring tissues and colonize distant organs is novel and first-in-the-field. (5) The concept of pharmacological inhibition of Jak2 signaling as a therapeutic strategy to broadly suppress metastatic progression of PC during Apalutamide treatment has high translational significance for therapy development for PC patients. (6) The concept of using the Jak2 inhibitors as a strategy to block metastatic progression of CRPC is a novel therapeutic approach for lethal Apalutamide-resistant PC. (7) The concept of pharmacological inhibition of Jak2 signaling as a therapeutic strategy to broadly suppress the expression of FL-AR and diverse AR-V species and proliferative and metastatic PC cell phenotype has high translational significance for therapy development for PC patients. (7) Jak2 control of AR expression in PC provides a conceptual advance that Jak2 inhibitors could represent “3rd generation anti-androgens” with broader and more durable effects on AR signaling than the 2nd generation agent Apalutamide.(8) The success with the proposed work is expected to lead to identification of the most potent Jak2 inhibitors for blocking Apalutamide-resistant PC growth and to a first-in-the-field phase I/II clinical trial to test the efficacy of the selected Jak2 inhibitors in suppressing growth of Apalutamide-resistant CRPC.

### Example 7A: Determine Whether Clinical Grade Apalutamide Induces Hyperactivated Jak2-Stat5 Signaling Pathway and Elevated AR Expression in Prostate Cancer

Study 7A.1.

We will determine if clinical grade Apalutamide induces activation of Jak2-Stat5 signaling pathway and AR levels in PC cell lines. We will evaluate if Apalutamide induces Jak2-Stat5 activation in PC cell lines (Table 2) in vitro and whether Apalutamide-induced Jak2-Stat5 signaling pathway activation in PC cells is dependent on AR (lentiviral shRNA knockdown of AR vs. lenti-shCtrl) and Jak2 (lentiviral shRNA knockdown of Jak2 vs. lenti-shCtrl). First, PC cell lines will be treated with increasing concentrations of Apalutamide vs. vehicle vs. Enzalutamide for 5, 10 and 15 days. Jak2 and Stat5 will be immunoprecipitated (IP:ed) from the cell lysates and blotted for active Jak2, active Stat5, Jak2 levels, AR levels and Stat5 levels with Actin blotting of the whole cell lysates (WCLs) as loading control. FL-AR and AR-V2 mRNA levels will be evaluated by qRT-PCR. Second, AR or Jak2 will genetically suppressed by lentiviral expression of shAR or shJak2, respectively, followed by Apalutamide treatment for 7 days. Jak2 and Stat5 will be immunoprecipitated (IP:ed) from the cell lysates and blotted for active Jak2, active Stat5, Jak2, AR and Stat5 levels with Actin blotting of the whole cell lysates (WCLs) as loading control. FL-AR and AR-V2 mRNA levels will be evaluated by qRT-PCR. We expect that Apalutamide induces phosphorylation and activation of both Jak2 and Stat5 in PC cells in vitro in conjunction with AR mRNA and protein levels, which will be blocked by genetic knockdown of the AR and Jak2.

Study 7A.2. Determine if Clinical Grade Apalutamide Induces Activation of Jak2-Stat5 Signaling Pathway and AR Levels in PC Cell Lines and in Patient-Derived PCs Ex Vivo in Tumor Explant Cultures.

We will evaluate if Apalutamide induces activation of Jak2-Stat5 signaling and AR mRNA/protein levels in patient-derived PCs (n=20) ex vivo in tumor explant cultures12, 13, 37, 40, 62, 86-91. Patient-derived clinical PCs will be cultured ex vivo in tumor explant cultures for seven and 14 days, as we have described previously. Tumor explants will be treated with Apalutamide at here different concentrations vs. vehicle at increasing concentrations with Prl treated group as control. Tissue morphology at the end of the culture will be monitored to ensure proper responses and tissue viability. Stat5 activation, AR mRNA and protein levels will be quantified by qPCR, immunoblotting (IB) and quantitative immunohistochemistry utilizing multiplex immunofluorescence histocytometry using Tissue Studio image analysis software. Statistics: Analysis of variance followed by Tukey-Kramer test will be used to test for differences among treatments with respect to logit-transformed fraction of positive cells. We expect that Apalutamide will induce Stat5 activation and AR mRNA and protein expression in patient-derived PC tissue explants.

### Example 7B: Identify the Top Performing Set of Jak2 Inhibitors in Blocking Jak2-Stat5 Signaling, AR Expression and PC Growth In Vitro in Apalutamide-Resistant Setting

Study 7B.1.

We will identify the most efficacious pharmacological Jak2 inhibitors in inducing death of PC cells in Apalutamide resistant setting alone as second line treatment vs. in combination with Apalutamide.

a) Second-line treatment alone: PC cell lines (Table 2) will be treated with Apalutamide for 20 days followed by pharmacological suppression of Jak2 by Jak2 inhibitors (Table 1) for 7 days. Jak2 and Stat5 will be inhibited in control cells by lentiviral expression of shJak2 or shStat5. Each inhibitor will be tested in 3 different concentrations. The fraction of surviving cells will be determined by MTT assay, FACS and crystal violet staining coupled with counting of the viable cells, as we have demonstrated previously. Levels of Stat5 activation will be evaluated by immunoprecipitation and immunoblotting. AR mRNA and protein levels will be evaluated by q-PCR and immunoblotting, respectively. The tested Jak2 inhibitors will be rank-ordered based on their efficacy in suppressing Stat5 signaling and inducing death of PC cells.

b) Second line treatment in combination with Apalutamide: PC cells (Table 2) will be treated by APA for 20 days followed by pharmacological suppression of Jak2 by Jak2 inhibitors (Table 1) for 7 days in combination with Apalutamide. Jak2 and Stat5 will be inhibited in control cells by lentiviral expression of shJak2 or shStat5. Each inhibitor will be tested in 3 different concentrations. The fraction of surviving cells will be determined by MTT assay, FACS and crystal violet staining coupled with counting of the viable cells as we have demonstrated previously. Levels of Stat5 activation will be evaluated by immunoprecipitation and immunoblotting. AR mRNA and protein levels will be evaluated by q-PCR and immunoblotting, respectively. The tested Jak2 inhibitors will be rank-ordered based on their efficacy in suppressing Stat5 signaling and inducing death of PC cells.

TABLE 1
Summary of JAK1/2 inhibitors to be tested.

| | | | |
|---|---|---|---|
| Compound Name | Target | Clinical Indications | Citations |

Approved:

| | | | |
|---|---|---|---|
| Baricitinib (trade | JAK1/JAK2 | Rheumatoid Arthritis | 74 |
| name Olumiant) | | (RA) | |
| Ruxolitinib (trade | JAK1/JAK2 | Psoriasis, | 75 |
| names Jakafi/Jakavi) | | Myelofibrosis, RA, | |
| | | PV | |
| Fedratinib | JAK2 | Myelofibrosis | 76 |
| (SAR302503; trade | FDA-approved | | |
| name Inrebic) | | | |

In Trials:

| | | | |
|---|---|---|---|
| Gandotinib (LY- | JAK2 | Myeloproliferative | 77 |
| 2784544) | | neoplasm | |
| Lestaurtinib (CEP- | JAK2 | Acute Myeloid | 78 |
| 701) | | leukemia | |
| Pacritinib (SB1518) | JAK2 | Relapse lymphoma, | 79 |
| | | advanced myeloid | |
| | | malignancies, MF | |

TABLE 2
PC cell lines, metastases models and patient-derived models.

1. CWR22Rv1; LAPC4; CWR22Pc; VCaP, LNCaP
2. 40 patient-derived PCs cultured ex vivo
3. CWR22Pc or LAPC4 xenograft PC tumor models for in vivo
tumor growth in nude mice

### Example 7C: Evaluate the Efficacy of the Top-Performing Jak2 Inhibitors in Blocking AR Expression and Growth of Apalutamide Resistant Human PC In Vivo in Xenograft Tumor Models Grown in Mice

Study 7C.1.

We will identify the most efficacious pharmacological Jak2 inhibitors in blocking Apalutamide resistant PC tumor growth in vivo in mice either alone as second line treatment or in combination with Apalutamide (a) or preventing development of Apalutamide-resistant PC xenograft tumor growth (b).

a) Second line treatment alone or in combination with Apalutamide for established Apalutamide-resistant PC xenograft tumors: We will determine the efficacy of the top Jak2 inhibitor in suppressing PC xenograft tumor growth when combined with Apalutamide. The most sensitive PC cell line identified in Aim 2 (CWR22Pc/LAPC4) will be grown subcutaneously (s.c.) in athymic nude mice until tumors form (appr. 25 days), followed by treatment with with Apalutamide vs. vehicle. Once the Apalutamide-resistance develops (around days 15-20—an estimate based on our previous tumor growth experiments using Enzalutamide⁶⁴), the mice will be divided to 4 groups (each n=10):

1) Vehicle (10 mice)
2) Apalutamide (60 mice)>>>>>
1) Vehicle (10 mice),
2) Apalutamide (10 mice),
3) Jak2 inhibitor (A) dose #1 (MTD) (10 mice),
4) Jak2-inhibitor (A) dose #2 (50% of the MTD) (10 mice),
6) Apalutamide+Jak2 inhibitor (A) dose #1 (MTD) (10 mice),
7) Apalutamide+Jak2 inhibitor (A) dose #2 (50% of the MTD) (10 mice).
   Timeline: Formation of tumors (25 d)>>>Development of APA-R (20 d)>>>>>>Treatment of APA-Resistant tumors (40 d)

The second-line treatment will last for approximately 40 days. The growth of the tumors will be monitored and recorded. At the end of the experiment, the tumors will be evaluated for the levels of active Stat5 by quantitative immunohistochemistry utilizing multiplex immunofluorescence histocytometry using Tissue Studio image analysis software. AR mRNA and protein levels will be determined by qPCR and immunoblotting. Statistics: Mixed effects linear regression will be used to model the trajectory of tumor growth over time. Tumor volumes will be log-transformed in order to satisfy the assumption of normality. Separate slopes and intercepts will be assumed for each group, and a random intercept term will be included to account for correlation among repeated measurements from the same mouse. We expect to identify the Jak2 inhibitors with the highest efficacy in blocking Apalutamide-resistant PC growth.

b) Evaluate the efficacy of pre-emptive combination therapy of Apalutamide with the top Jak2 inhibitor in preventing development of APA-resistant CRPC xenograft tumor growth when administered in combination with APA (Phase I):

PC cells (CWR22Pc) will be injected s.c. in nude mice supplemented with DHT pellets. Once the androgen-driven tumors form (6-7 mm in diameter) (Phase I) (on day 25), the mice will be treated daily for 60 days with vehicle or APA (30 mg/kg; p.o.) alone or in combination with the top Jak2 inhibitor (2 doses). Each treatment group includes 10 mice based on the power calculations. At the time of sacrifice, the tumors will be evaluated for AR and Stat5 protein levels by IHC and IB, and for FL-AR and AR-V mRNA levels by quantitative RT-PCR. The tumors will be analyzed for apoptosis and proliferative indexes by TUNEL and PCNA/Ki67 IHC (Tissue Studio Analysis), respectively37, and changes in tumor volumes will be measured as previously described. We expect that the top Jak2 inhibitor will significantly suppress AR levels and development of APA-resistant PC tumors compared to the other treatment groups.

Timeline: Tumor formation (25 d)>>>Treatment to prevent development of APA-resistance (60 d)
1) Vehicle (10 mice),
2) Apalutamide (10 mice),
3) Apalutamide+Jak2 inhibitor (A) dose #1 (MTD) (10 mice),
4) Apalutamide+Jak2 inhibitor (A) dose #2 (50% of the MTD) (10 mice).

Study 7C.3. We Will Evaluate the Efficacy of the Top 3 Jak2 Inhibitors in Inducing Death of PC Epithelium and Stroma that Survives Apalutamide Treatment in Clinical Patient-Derived PCs in Tumor Explant Cultures (n=15).

Individual patient-derived PCs will be cultured with vehicle vs. Apalutamide for 10 days followed by treatment with the top 2 Jak2 inhibitors at three different concentrations for 5 days. Viable epithelial and stromal cells will be evaluated by histology and the levels of active Stat5 will be determined by immunohistochemistry of active Stat5. Statistics: For PC tumor explant cultures, mixed effects linear regression will be used to compare groups with respect to mean outcomes (cell viability, Stat5 positive cells). A random intercept term will be used to account for clustering by explant culture. Pairwise comparisons will be used to evaluate each dose group and compare to the control. P-values for the pairwise comparisons will be adjusted using the Bonferroni method. We will also determine the Stat5 activation status of the individual PCs prior to the culture to evaluate if the Stat5 status will serve a predictive marker for patient selection for Jak2-inhibitor therapy.

### Example 7D: Evaluate the Predominant Transcriptomes in the Residual Tumors Surviving Jak2-Inhibitor Treatment in the Apalutamide-Resistant Setting In Vivo

RNA will be pooled from the residual CWR22Pc tumors surviving Jak2 inhibitor treatment vs. vehicle treated tumors The RNA will be converted to stranded RNA-seq libraries and sequenced on an Illumina NextSeq instrument at 2×75 bp settings to a targeted depth of 100 million reads per sample. The goal of RNA-seq data analysis will be to test the hypothesis that Jak2 inhibition perturbs a similar set of genes as AR inhibition, which is expected from our data showing that Jak2-Stat5 drives AR expression in PC. For this, RNA-seq reads will be mapped to the human reference genome, transcripts will be assembled, and differential expression will be assessed as per our previous work. We will use gene set enrichment analysis (GSEA)⁹⁵ to test whether a set of 100-200 genes activated by AR displays positive enrichment in the RNA-seq gene expression datasets derived from cells with active vs. repressed Jak2. Similarly, we will test whether a set of 100-200 genes repressed by AR displays negative enrichment in the RNA-seq gene expression data sets derived from cells with active vs. repressed Jak2/Stat5. We will also perform the reciprocal comparisons with the top 100-200 Jak2-Stat5-activated and—repressed genes. If the anticipated positive/negative enrichments are observed, we will then develop a consolidated Z-score to represent the expression of these gene sets and use t-tests to compare their expression under Jak2 inhibition vs. non-inhibited conditions.

### REFERENCES

1. Attard G, Parker C, Eeles R A, Schroder F, Tomlins S A, Tannock I, et al. Prostate cancer. Lancet. 2016; 387(10013):70-82.
2. Attard G, Cooper C S, de Bono J S. Steroid hormone receptors in prostate cancer: a hard habit to break? Cancer Cell. 2009; 16(6):458-62.
3. Beer T M, Armstrong A J, Rathkopf D E, Loriot Y, Sternberg C N, Higano C S, et al. Enzalutamide in metastatic prostate cancer before chemotherapy. N Engl J Med. 2014; 371(5):424-33.
4. Ryan C J, Smith M R, de Bono J S, Molina A, Logothetis C J, de Souza P, et al.

Abiraterone in Metastatic Prostate Cancer without Previous Chemotherapy. N Engl J Med. 2012; 368:138-48.
5. Watson P A, Arora V K, Sawyers C L. Emerging mechanisms of resistance to androgen receptor inhibitors in prostate cancer. Nat Rev Cancer. 2015; 15(12):701-11.
6. Hoang D T, Gu L, Liao Z, Shen F, Talati P G, Koptyra M, et al. Inhibition of Stat5a/b Enhances Proteasomal Degradation of Androgen Receptor Liganded by Antiandrogens in Prostate Cancer. Mol Cancer Ther. 2015; 14(3):713-26.
7. Gu L, Liao Z, Hoang D T, Dagvadorj A, Gupta S, Blackmon S, et al. Pharmacologic Inhibition of Jak2-Stat5 Signaling By Jak2 Inhibitor AZD1480 Potently Suppresses Growth of Both Primary and Castrate-Resistant Prostate Cancer. Clin Cancer Res. 2013; 19(20):5658-74.
8. Shodeinde A L, Barton B E. Potential use of STAT3 inhibitors in targeted prostate cancer therapy: future prospects. Onco Targets Ther. 2012; 5:119-25.
9. Wong Y N, Ferraldeschi R, Attard G, de Bono J. Evolution of androgen receptor targeted therapy for advanced prostate cancer. Nat Rev Clin Oncol. 2014; 11(6):365-76.
10. Balk S P, Knudsen K E. AR, the cell cycle, and prostate cancer. Nucl Recept Signal. 2008; 6:e001.
11. He B, Bai S, Hnat A T, Kalman R I, Minges J T, Patterson C, et al. An androgen receptor NH2-terminal conserved motif interacts with the COOH terminus of the Hsp70-interacting protein (CHIP). J Biol Chem. 2004; 279(29):30643-53.
12. Gelmann E P. Molecular biology of the androgen receptor. J Clin Oncol. 2002; 20(13):3001-15.
13. Chen C D, Welsbie D S, Tran C, Baek S H, Chen R, Vessella R, et al. Molecular determinants of resistance to antiandrogen therapy. Nat Med. 2004; 10(1):33-9.
14. Robinson D, Van Allen E M, Wu Y M, Schultz N, Lonigro R J, Mosquera J M, et al. Integrative clinical genomics of advanced prostate cancer. Cell. 2015; 161(5):1215-28.
15. Henzler C, Li Y, Yang R, McBride T, Ho Y, Sprenger C, et al. Truncation and constitutive activation of the androgen receptor by diverse genomic rearrangements in prostate cancer. Nat Commun. 2016; 7:13668.
16. Van Etten J L, Nyquist M, Li Y, Yang R, Ho Y, Johnson R, et al. Targeting a Single Alternative Polyadenylation Site Coordinately Blocks Expression of Androgen Receptor mRNA Splice Variants in Prostate Cancer. Cancer Res. 2017; 77(19):5228-35.
17. Kohli M, Ho Y, Hillman D W, Van Etten J L, Henzler C, Yang R, et al. Androgen receptor variant AR-V9 is co-expressed with AR-V7 in prostate cancer metastases and predicts abiraterone resistance. Clin Cancer Res. 2017; 23:4704-15.
18. Li H, Ahonen T J, Alanen K, Xie J, LeBaron M J, Pretlow T G, et al. Activation of signal transducer and activator of transcription 5 in human prostate cancer is associated with high histological grade. Cancer Res. 2004; 64(14):4774-82.
19. Dagvadorj A, Collins S, Jomain J B, Abdulghani J, Karras J, Zellweger T, et al. Autocrine prolactin promotes prostate cancer cell growth via Janus kinase-2-signal transducer and activator of transcription-5a/b signaling pathway. Endocrinology. 2007; 148(7):3089-101.
20. Silvennoinen O, Hubbard S R. Molecular insights into regulation of JAK2 in myeloproliferative neoplasms. Blood. 2015; 125(22):3388-92.
21. Ghoreschi K, Laurence A, O'Shea J J. Janus kinases in immune cell signaling. Immunol Rev. 2009; 228(1):273-87.
22. Rui H, Djeu J Y, Evans G A, Kelly P A, Farrar W L. Prolactin receptor triggering. Evidence for rapid tyrosine kinase activation. J Biol Chem. 1992; 267(33):24076-81.
23. Rui H, Lebrun J J, Kirken R A, Kelly P A, Farrar W L. JAK2 activation and cell proliferation induced by antibody-mediated prolactin receptor dimerization. Endocrinology. 1994; 135(4):1299-306.
24. Talati P G, Gu L, Ellsworth E M, Girondo M A, Trerotola M, Hoang D T, et al. Jak2-Stat5a/b Signaling Induces Epithelial-to-Mesenchymal Transition and Stem-Like Cell Properties in Prostate Cancer. Am J Pathol. 2015; 185(9):2505-22.
25. Levy D E, Darnell J E, Jr. Stats: transcriptional control and biological impact. Nat Rev Mol Cell Biol. 2002; 3(9):651-62.
26. Quintas-Cardama A, Verstovsek S. Molecular pathways: Jak/STAT pathway:
   mutations, inhibitors, and resistance. Clin Cancer Res. 2013; 19(8):1933-40.
27. Ihle J N. The Stat family in cytokine signaling. Curr Opin Cell Biol. 2001; 13(2):211-7.
28. Liu X, Robinson G W, Gouilleux F, Groner B, Hennighausen L. Cloning and expression of Stat5 and an additional homologue (Stat5b) involved in prolactin signal transduction in mouse mammary tissue. Proc Natl Acad Sci USA. 1995; 92(19):8831-5.
29. Liu X, Robinson G W, Hennighausen L. Activation of Stat5a and Stat5b by tyrosine phosphorylation is tightly linked to mammary gland differentiation. Mol Endocrinol. 1996; 10(12):1496-506.
30. Mesa R A, Vannucchi A M, Mead A, Egyed M, Szoke A, Suvorov A, et al. Pacritinib versus best available therapy for the treatment of myelofibrosis irrespective of baseline cytopenias (PERSIST-1): an international, randomised, phase 3 trial. Lancet Haematol. 2017; 4(5):e225-e36.
31. Verstovsek S, Odenike O, Singer J W, Granston T, Al-Fayoumi S, Deeg H J. Phase 1/2 study of pacritinib, a next generation JAK2/FLT3 inhibitor, in myelofibrosis or other myeloid malignancies. J Hematol Oncol. 2016; 9(1):137.
32. Jain T, Mesa R. The development, safety and efficacy of pacritinib for the treatment of myelofibrosis. Expert Rev Anticancer Ther. 2016; 16(11):1101-8.
33. Bose P, Alfayez M, Verstovsek S. New Concepts of Treatment for Patients with Myelofibrosis. Curr Treat Options Oncol. 2019; 20(1):5.
34. Passamonti F, Maffioli M. The role of JAK2 inhibitors in MPNs 7 years after approval. Blood. 2018; 131(22):2426-35.
35. Ahonen T J, Xie J, LeBaron M J, Zhu J, Nurmi M, Alanen K, et al. Inhibition of transcription factor Stat5 induces cell death of human prostate cancer cells. J Biol Chem. 2003; 278(29):27287-92.
36. Dagvadorj A, Kirken R A, Leiby B, Karras J, Nevalainen M T. Transcription factor signal transducer and activator of transcription 5 promotes growth of human prostate cancer cells in vivo. Clin Cancer Res. 2008; 14(5):1317-24.
37. Gu L, Dagvadorj A, Lutz J, Leiby B, Bonuccelli G, Lisanti M P, et al. Transcription factor Stat3 stimulates metastatic behavior of human prostate cancer cells in vivo, whereas Stat5b has a preferential role in the promotion of prostate cancer cell viability and tumor growth. Am J Pathol. 2010; 176(4):1959-72.
38. Gu L, Vogiatzi P, Puhr M, Dagvadorj A, Lutz J, Ryder A, et al. Stat5 promotes metastatic behavior of human prostate cancer cells in vitro and in vivo. Endocr Relat Cancer. 2010; 17(2):481-93.
39. Liao Z, Gu L, Vergalli J, Mariani S A, De Dominici M, Lokareddy R K, et al. Structure-Based Screen Identifies a Potent Small Molecule Inhibitor of Stat5a/b with Therapeutic Potential for Prostate Cancer and Chronic Myeloid Leukemia. Mol Cancer Ther. 2015; 14(8):1777-93.
40. Gu L Z X, Visakorpi T, Alanen K, Mirtti T, Edmonston T, Nevalainen M T. Activating mutation (V617F) in the tyrosine kinase Jak2 is absent in local-confined and castration-resistant prostate cancer. Cellular Oncology. 2010; 33:55-59.
41. Hoang D T, Iczkowski K A, Kilari D, See W, Nevalainen M T. Androgen receptor-dependent and -independent mechanisms driving prostate cancer progression: Opportunities for therapeutic targeting from multiple angles. Oncotarget. 2017; 8(2):3724-45.
42. Kazansky A V, Spencer D M, Greenberg N M. Activation of signal transducer and activator of transcription 5 is required for progression of autochthonous prostate cancer: evidence from the transgenic adenocarcinoma of the mouse prostate system. Cancer Res. 2003; 63(24):8757-62.
43. Thomas C, Zoubeidi A, Kuruma H, Fazli L, Lamoureux F, Beraldi E, et al. Transcription factor Stat5 knockdown enhances androgen receptor degradation and delays castration-resistant prostate cancer progression in vivo. Mol Cancer Ther. 2011; 10(2):347-59.
44. Haddad B R, Gu L, Mirtti T, Dagvadorj A, Vogiatzi P, Hoang D T, et al. STAT5A/B gene locus undergoes amplification during human prostate cancer progression. Am J Pathol. 2013; 182(6):2264-75.
45. Li H, Zhang Y, Glass A, Zellweger T, Gehan E, Bubendorf L, et al. Activation of signal transducer and activator of transcription-5 in prostate cancer predicts early recurrence. Clin Cancer Res. 2005; 11(16):5863-8.
46. Mirtti T, Leiby B E, Abdulghani J, Aaltonen E, Pavela M, Mamtani A, et al. Nuclear Stat5a/b predicts early recurrence and prostate cancer-specific death in patients treated by radical prostatectomy. Hum Pathol. 2013; 44(3):310-9.
47. Li Y, Alsagabi M, Fan D, Bova G S, Tewfik A H, Dehm S M. Intragenic rearrangement and altered RNA splicing of the androgen receptor in a cell-based model of prostate cancer progression. Cancer Res. 2011; 71(6):2108-17.
48. Ahonen T J, Harkonen P L, Rui H, Nevalainen M T. PRL signal transduction in the epithelial compartment of rat prostate maintained as long-term organ cultures in vitro. Endocrinology. 2002; 143(1):228-38.
49. Nevalainen M T, Xie J, Bubendorf L, Wagner K U, Rui H. Basal activation of transcription factor signal transducer and activator of transcription (Stat5) in nonpregnant mouse and human breast epithelium. Mol Endocrinol. 2002; 16(5):1108-24.
50. Udhane V, Maranto C, Hoang D T, Gu L, Erickson A, Devi S, et al. Enzalutamide-Induced Feed-Forward Signaling Loop Promotes Therapy-Resistant Prostate Cancer Growth Providing an Exploitable Molecular Target for Jak2 Inhibitors. Mol Cancer Ther. 2020; 19(1):231-46.
51. Dagvadorj A, Tan S H, Liao Z, Cavalli L R, Haddad B R, Nevalainen M T. Androgen-regulated and highly tumorigenic human prostate cancer cell line established from a transplantable primary CWR22 tumor. Clin Cancer Res. 2008; 14(19):6062-72.
52. Maranto C, Udhane V, Hoang D T, Gu L, Alexeev V, Malas K, et al. STAT5A/B Blockade Sensitizes Prostate Cancer to Radiation through Inhibition of RAD51 and DNA Repair. Clin Cancer Res. 2018; 24(8):1917-31.
53. Nevalainen M T, Harkonen P L, Valve E M, Ping W, Nurmi M, Martikainen P M. Hormone regulation of human prostate in organ culture. Cancer Res. 1993; 53(21):5199-207.
54. Nevalainen M T, Valve E M, Ingleton P M, Nurmi M, Martikainen P M, Harkonen P L. Prolactin and prolactin receptors are expressed and functioning in human prostate. J Clin Invest. 1997; 99(4):618-27.

1. A method of inhibiting and reducing expression of androgen receptor (AR) and androgen receptor variants (AR variants) in a prostate cancer cell, the method comprising:
contacting the prostate cancer cell with an effective amount of a Jak2 inhibitor which inhibits through Stat5 pathway within the cell to inhibit or reduce AR and AR variant expression in the prostate cancer cell.

2. The method of claim 1, wherein the Jak2 inhibitor is selected from the group consisting of pacritinib, gandotinib, baricitinib, and fedratinib.

3. The method of claim 1, wherein the prostate cancer cell is selected from the group consisting of androgen-sensitive prostate cancer, advanced prostate cancer and castrate resistant prostate cancer (CRPC).

4. The method of claim 1, wherein the prostate cancer is resistant to treatment with a luteinizing hormone-releasing hormone (LHRH) agonist, enzalutamide, abiraterone, apalutamide, or darolutamide.

5. The method of claim 1, wherein the prostate cancer cell is within a subject having prostate cancer, and the contacting comprises administering a therapeutically effective amount of the Jak2 inhibitor to the subject.

6. The method of claim 5, wherein the subject has prostate cancer that is resistant to anti-androgen therapy.

7. The method of claim 5, wherein the subject has undergone one or more treatment for prostate cancer, and wherein the subject has developed resistance to that treatment.

8. The method of claim 6, wherein the treatment comprises enzalutamide, abiraterone, apalutamide, or darolutamide or medical castration.

9. The method of claim 5, wherein the subject is further administered an anti-androgen therapy.

10. The method of claim 5, wherein the Jak2 inhibitor is pacritinib or fedratinib.

11. A method of inhibiting or reducing growth of prostate cancer (PC) in a subject having PC, the method comprising administering a therapeutically effective amount of a Jak2 inhibitor that acts through the Stat5 pathway selected from the group consisting pacritinib, gandotinib, baricitinib, and fedratinib to inhibit or reduce growth of the PC cells within the subject.

12. The method of claim 11, wherein the PC is resistant to one or more anti-androgen therapies.

13. The method of claim 12, wherein the CRPC is resistant to treatment by enzalutamide or abiraterone.

14. The method of claim 11, wherein the method further comprises treating with one or more anti-androgen therapies.

15. The method of claim 11, wherein the cancer is castrate resistant prostate cancer (CRPC).

16. The method of claim 11, wherein the prostate cancer is organ-confined, hormone-sensitive prostate cancer, locally advanced prostate cancer, or metastasized prostate cancer.

17. A method of inhibiting anti-androgen resistant CRPC cell growth in a subject with prostate cancer, the method comprising administering an effective amount of a Jak2 inhibitor that inhibits through the Stat5 pathway to the subject with prostate cancer in order to treat the cancer and inhibit growth of anti-androgen-resistant prostate cancer cells.

18. The method of claim 17, wherein the method further comprises administering and an amount of an anti-androgen in combination with the Jak2 inhibitor.

19. The method of claim 18, wherein the anti-androgen is enzalutamide, abiraterone, apalutamide, or darolutamide.

20. The method of claim 17, wherein the Jak2 inhibitor is pacritinib, fedratinib, baricitinib or gandotinib.

21. The method of claim 17, wherein the subject has not undergone previous cancer treatment.
